SEQUENCE LISTING (284 sequences)

SEQ ID NO: 1 - RNA, 21 nt, synthetic construct
  source 1..21: mol_type = other RNA
caagcaaaaatgtgctagtcc

SEQ ID NO: 2 - RNA, 21 nt, synthetic construct
  source 1..21: mol_type = other RNA
caagcaaaaatgtgctagtac

SEQ ID NO: 3 - RNA, 21 nt, synthetic construct
  source 1..21: mol_type = other RNA
caagcaaaaatgtgctagtgg

SEQ ID NO: 4 - RNA, 21 nt, synthetic construct
  source 1..21: mol_type = other RNA
caagcaaaaatgtgctagtga

SEQ ID NO: 5 - RNA, 21 nt, synthetic construct
  source 1..21: mol_type = other RNA
caagcaaaaatgtgctagcgc

SEQ ID NO: 6 - RNA, 21 nt, synthetic construct
  source 1..21: mol_type = other RNA
caagcaaaaatgtgctagggc

SEQ ID NO: 7 - RNA, 21 nt, synthetic construct
  source 1..21: mol_type = other RNA
gctagtaccaaaatcggccaa

SEQ ID NO: 8 - RNA, 21 nt, synthetic construct
  source 1..21: mol_type = other RNA
gctagtcccaaaatcggccaa

SEQ ID NO: 9 - RNA, 21 nt, synthetic construct
  source 1..21: mol_type = other RNA
gctagtgacaaaatcggccaa

SEQ ID NO: 10 - RNA, 21 nt, synthetic construct
  source 1..21: mol_type = other RNA
gctagtggcaaaatcggccaa

SEQ ID NO: 11 - RNA, 21 nt, synthetic construct
  source 1..21: mol_type = other RNA
gctagggccaaaatcggccaa

SEQ ID NO: 12 - RNA, 21 nt, synthetic construct
  source 1..21: mol_type = other RNA
gctagcgccaaaatcggccaa

SEQ ID NO: 13 - RNA, 21 nt, synthetic construct
  source 1..21: mol_type = other RNA
ctgtgagcaatcacgtgtagt

SEQ ID NO: 14 - RNA, 20 nt, synthetic construct
  source 1..20: mol_type = other RNA
aaatgtgctagtaccaaaat

SEQ ID NO: 15 - RNA, 20 nt, synthetic construct
  source 1..20: mol_type = other RNA
aaatgtgctagtcccaaaat

SEQ ID NO: 16 - RNA, 20 nt, synthetic construct
  source 1..20: mol_type = other RNA
aaatgtgctagtgacaaaat

SEQ ID NO: 17 - RNA, 20 nt, synthetic construct
  source 1..20: mol_type = other RNA
aaatgtgctagtggcaaaat

SEQ ID NO: 18 - RNA, 20 nt, synthetic construct
  source 1..20: mol_type = other RNA
aaatgtgctagggccaaaat

SEQ ID NO: 19 - RNA, 20 nt, synthetic construct
  source 1..20: mol_type = other RNA
aaatgtgctagcgccaaaat

SEQ ID NO: 20 - RNA, 21 nt, synthetic construct
  source 1..21: mol_type = other RNA
caagcaaaaatgtgctagtgc

SEQ ID NO: 21 - RNA, 21 nt, synthetic construct
  source 1..21: mol_type = other RNA
gaagcaaaaatgtgctagtgc

SEQ ID NO: 22 - RNA, 21 nt, synthetic construct
  source 1..21: mol_type = other RNA
taagcaaaaatgtgctagtgc

SEQ ID NO: 23 - RNA, 21 nt, synthetic construct
  source 1..21: mol_type = other RNA
aaagcaaaaatgtgctagtgc

SEQ ID NO: 24 - RNA, 21 nt, synthetic construct
  source 1..21: mol_type = other RNA
ctagcaaaaatgtgctagtgc

SEQ ID NO: 25 - RNA, 21 nt, synthetic construct
  source 1..21: mol_type = other RNA
ccagcaaaaatgtgctagtgc

SEQ ID NO: 26 - RNA, 21 nt, synthetic construct
  source 1..21: mol_type = other RNA
cgagcaaaaatgtgctagtgc

SEQ ID NO: 27 - RNA, 21 nt, synthetic construct
  source 1..21: mol_type = other RNA
catgcaaaaatgtgctagtgc

SEQ ID NO: 28 - RNA, 21 nt, synthetic construct
  source 1..21: mol_type = other RNA
cacgcaaaaatgtgctagtgc

SEQ ID NO: 29 - RNA, 21 nt, synthetic construct
  source 1..21: mol_type = other RNA
caggcaaaaatgtgctagtgc

SEQ ID NO: 30 - RNA, 21 nt, synthetic construct
  source 1..21: mol_type = other RNA
caaacaaaaatgtgctagtgc

SEQ ID NO: 31 - RNA, 21 nt, synthetic construct
  source 1..21: mol_type = other RNA
caatcaaaaatgtgctagtgc

SEQ ID NO: 32 - RNA, 21 nt, synthetic construct
  source 1..21: mol_type = other RNA
caaccaaaaatgtgctagtgc

SEQ ID NO: 33 - RNA, 21 nt, synthetic construct
  source 1..21: mol_type = other RNA
caagaaaaaatgtgctagtgc

SEQ ID NO: 34 - RNA, 21 nt, synthetic construct
  source 1..21: mol_type = other RNA
caagtaaaaatgtgctagtgc

SEQ ID NO: 35 - RNA, 21 nt, synthetic construct
  source 1..21: mol_type = other RNA
caaggaaaaatgtgctagtgc

SEQ ID NO: 36 - RNA, 21 nt, synthetic construct
  source 1..21: mol_type = other RNA
caagctaaaatgtgctagtgc

SEQ ID NO: 37 - RNA, 21 nt, synthetic construct
  source 1..21: mol_type = other RNA
caagccaaaatgtgctagtgc

SEQ ID NO: 38 - RNA, 21 nt, synthetic construct
  source 1..21: mol_type = other RNA
caagcgaaaatgtgctagtgc

SEQ ID NO: 39 - RNA, 21 nt, synthetic construct
  source 1..21: mol_type = other RNA
caagcataaatgtgctagtgc

SEQ ID NO: 40 - RNA, 21 nt, synthetic construct
  source 1..21: mol_type = other RNA
caagcacaaatgtgctagtgc

SEQ ID NO: 41 - RNA, 21 nt, synthetic construct
  source 1..21: mol_type = other RNA
caagcagaaatgtgctagtgc

SEQ ID NO: 42 - RNA, 21 nt, synthetic construct
  source 1..21: mol_type = other RNA
caagcaataatgtgctagtgc

SEQ ID NO: 43 - RNA, 21 nt, synthetic construct
  source 1..21: mol_type = other RNA
caagcaacaatgtgctagtgc

SEQ ID NO: 44 - RNA, 21 nt, synthetic construct
  source 1..21: mol_type = other RNA
caagcaagaatgtgctagtgc

SEQ ID NO: 45 - RNA, 21 nt, synthetic construct
  source 1..21: mol_type = other RNA
caagcaaatatgtgctagtgc

SEQ ID NO: 46 - RNA, 21 nt, synthetic construct
  source 1..21: mol_type = other RNA
caagcaaacatgtgctagtgc

SEQ ID NO: 47 - RNA, 21 nt, synthetic construct
  source 1..21: mol_type = other RNA
caagcaaagatgtgctagtgc

SEQ ID NO: 48 - RNA, 21 nt, synthetic construct
  source 1..21: mol_type = other RNA
caagcaaaattgtgctagtgc

SEQ ID NO: 49 - RNA, 21 nt, synthetic construct
  source 1..21: mol_type = other RNA
caagcaaaactgtgctagtgc

SEQ ID NO: 50 - RNA, 21 nt, synthetic construct
  source 1..21: mol_type = other RNA
caagcaaaagtgtgctagtgc

SEQ ID NO: 51 - RNA, 21 nt, synthetic construct
  source 1..21: mol_type = other RNA
caagcaaaaaagtgctagtgc

SEQ ID NO: 52 - RNA, 21 nt, synthetic construct
  source 1..21: mol_type = other RNA
caagcaaaaacgtgctagtgc

SEQ ID NO: 53 - RNA, 21 nt, synthetic construct
  source 1..21: mol_type = other RNA
caagcaaaaaggtgctagtgc

SEQ ID NO: 54 - RNA, 21 nt, synthetic construct
  source 1..21: mol_type = other RNA
caagcaaaaatatgctagtgc

SEQ ID NO: 55 - RNA, 21 nt, synthetic construct
  source 1..21: mol_type = other RNA
caagcaaaaatttgctagtgc

SEQ ID NO: 56 - RNA, 21 nt, synthetic construct
  source 1..21: mol_type = other RNA
caagcaaaaatctgctagtgc

SEQ ID NO: 57 - RNA, 21 nt, synthetic construct
  source 1..21: mol_type = other RNA
caagcaaaaatgagctagtgc

SEQ ID NO: 58 - RNA, 21 nt, synthetic construct
  source 1..21: mol_type = other RNA
caagcaaaaatgcgctagtgc

SEQ ID NO: 59 - RNA, 21 nt, synthetic construct
  source 1..21: mol_type = other RNA
caagcaaaaatgggctagtgc

SEQ ID NO: 60 - RNA, 21 nt, synthetic construct
  source 1..21: mol_type = other RNA
caagcaaaaatgtactagtgc

SEQ ID NO: 61 - RNA, 21 nt, synthetic construct
  source 1..21: mol_type = other RNA
caagcaaaaatgttctagtgc

SEQ ID NO: 62 - RNA, 21 nt, synthetic construct
  source 1..21: mol_type = other RNA
caagcaaaaatgtcctagtgc

SEQ ID NO: 63 - RNA, 21 nt, synthetic construct
  source 1..21: mol_type = other RNA
caagcaaaaatgtgatagtgc

SEQ ID NO: 64 - RNA, 21 nt, synthetic construct
  source 1..21: mol_type = other RNA
caagcaaaaatgtgttagtgc

SEQ ID NO: 65 - RNA, 21 nt, synthetic construct
  source 1..21: mol_type = other RNA
caagcaaaaatgtggtagtgc

SEQ ID NO: 66 - RNA, 21 nt, synthetic construct
  source 1..21: mol_type = other RNA
caagcaaaaatgtgcaagtgc

SEQ ID NO: 67 - RNA, 21 nt, synthetic construct
  source 1..21: mol_type = other RNA
caagcaaaaatgtgccagtgc

SEQ ID NO: 68 - RNA, 21 nt, synthetic construct
  source 1..21: mol_type = other RNA
caagcaaaaatgtgcgagtgc

SEQ ID NO: 69 - RNA, 21 nt, synthetic construct
  source 1..21: mol_type = other RNA
caagcaaaaatgtgcttgtgc

SEQ ID NO: 70 - RNA, 21 nt, synthetic construct
  source 1..21: mol_type = other RNA
caagcaaaaatgtgctcgtgc

SEQ ID NO: 71 - RNA, 21 nt, synthetic construct
  source 1..21: mol_type = other RNA
caagcaaaaatgtgctggtgc

SEQ ID NO: 72 - RNA, 21 nt, synthetic construct
  source 1..21: mol_type = other RNA
caagcaaaaatgtgctaatgc

SEQ ID NO: 73 - RNA, 21 nt, synthetic construct
  source 1..21: mol_type = other RNA
caagcaaaaatgtgctattgc

SEQ ID NO: 74 - RNA, 21 nt, synthetic construct
  source 1..21: mol_type = other RNA
caagcaaaaatgtgctactgc

SEQ ID NO: 75 - RNA, 20 nt, synthetic construct
  source 1..20: mol_type = other RNA
aaatgtgctagtgccaaaat

SEQ ID NO: 76 - RNA, 20 nt, synthetic construct
  source 1..20: mol_type = other RNA
taatgtgctagtgccaaaat

SEQ ID NO: 77 - RNA, 20 nt, synthetic construct
  source 1..20: mol_type = other RNA
caatgtgctagtgccaaaat

SEQ ID NO: 78 - RNA, 20 nt, synthetic construct
  source 1..20: mol_type = other RNA
gaatgtgctagtgccaaaat

SEQ ID NO: 79 - RNA, 20 nt, synthetic construct
  source 1..20: mol_type = other RNA
atatgtgctagtgccaaaat

SEQ ID NO: 80 - RNA, 20 nt, synthetic construct
  source 1..20: mol_type = other RNA
acatgtgctagtgccaaaat

SEQ ID NO: 81 - RNA, 20 nt, synthetic construct
  source 1..20: mol_type = other RNA
agatgtgctagtgccaaaat

SEQ ID NO: 82 - RNA, 20 nt, synthetic construct
  source 1..20: mol_type = other RNA
aattgtgctagtgccaaaat

SEQ ID NO: 83 - RNA, 20 nt, synthetic construct
  source 1..20: mol_type = other RNA
aactgtgctagtgccaaaat

SEQ ID NO: 84 - RNA, 20 nt, synthetic construct
  source 1..20: mol_type = other RNA
aagtgtgctagtgccaaaat

SEQ ID NO: 85 - RNA, 20 nt, synthetic construct
  source 1..20: mol_type = other RNA
aaaagtgctagtgccaaaat

SEQ ID NO: 86 - RNA, 20 nt, synthetic construct
  source 1..20: mol_type = other RNA
aaacgtgctagtgccaaaat

SEQ ID NO: 87 - RNA, 20 nt, synthetic construct
  source 1..20: mol_type = other RNA
aaaggtgctagtgccaaaat

SEQ ID NO: 88 - RNA, 20 nt, synthetic construct
  source 1..20: mol_type = other RNA
aaatatgctagtgccaaaat

SEQ ID NO: 89 - RNA, 20 nt, synthetic construct
  source 1..20: mol_type = other RNA
aaatttgctagtgccaaaat

SEQ ID NO: 90 - RNA, 20 nt, synthetic construct
  source 1..20: mol_type = other RNA
aaatctgctagtgccaaaat

SEQ ID NO: 91 - RNA, 20 nt, synthetic construct
  source 1..20: mol_type = other RNA
aaatgagctagtgccaaaat

SEQ ID NO: 92 - RNA, 20 nt, synthetic construct
  source 1..20: mol_type = other RNA
aaatgcgctagtgccaaaat

SEQ ID NO: 93 - RNA, 20 nt, synthetic construct
  source 1..20: mol_type = other RNA
aaatgggctagtgccaaaat

SEQ ID NO: 94 - RNA, 20 nt, synthetic construct
  source 1..20: mol_type = other RNA
aaatgtactagtgccaaaat

SEQ ID NO: 95 - RNA, 20 nt, synthetic construct
  source 1..20: mol_type = other RNA
aaatgttctagtgccaaaat

SEQ ID NO: 96 - RNA, 20 nt, synthetic construct
  source 1..20: mol_type = other RNA
aaatgtcctagtgccaaaat

SEQ ID NO: 97 - RNA, 20 nt, synthetic construct
  source 1..20: mol_type = other RNA
aaatgtgatagtgccaaaat

SEQ ID NO: 98 - RNA, 20 nt, synthetic construct
  source 1..20: mol_type = other RNA
aaatgtgttagtgccaaaat

SEQ ID NO: 99 - RNA, 20 nt, synthetic construct
  source 1..20: mol_type = other RNA
aaatgtggtagtgccaaaat

SEQ ID NO: 100 - RNA, 20 nt, synthetic construct
  source 1..20: mol_type = other RNA
aaatgtgcaagtgccaaaat

SEQ ID NO: 101 - RNA, 20 nt, synthetic construct
  source 1..20: mol_type = other RNA
aaatgtgccagtgccaaaat

SEQ ID NO: 102 - RNA, 20 nt, synthetic construct
  source 1..20: mol_type = other RNA
aaatgtgcgagtgccaaaat

SEQ ID NO: 103 - RNA, 20 nt, synthetic construct
  source 1..20: mol_type = other RNA
aaatgtgcttgtgccaaaat

SEQ ID NO: 104 - RNA, 20 nt, synthetic construct
  source 1..20: mol_type = other RNA
aaatgtgctcgtgccaaaat

SEQ ID NO: 105 - RNA, 20 nt, synthetic construct
  source 1..20: mol_type = other RNA
aaatgtgctggtgccaaaat

SEQ ID NO: 106 - RNA, 20 nt, synthetic construct
  source 1..20: mol_type = other RNA
aaatgtgctaatgccaaaat

SEQ ID NO: 107 - RNA, 20 nt, synthetic construct
  source 1..20: mol_type = other RNA
aaatgtgctattgccaaaat

SEQ ID NO: 108 - RNA, 20 nt, synthetic construct
  source 1..20: mol_type = other RNA
aaatgtgctactgccaaaat

SEQ ID NO: 109 - RNA, 20 nt, synthetic construct
  source 1..20: mol_type = other RNA
aaatgtgctagtgcaaaaat

SEQ ID NO: 110 - RNA, 20 nt, synthetic construct
  source 1..20: mol_type = other RNA
aaatgtgctagtgctaaaat

SEQ ID NO: 111 - RNA, 20 nt, synthetic construct
  source 1..20: mol_type = other RNA
aaatgtgctagtgcgaaaat

SEQ ID NO: 112 - RNA, 20 nt, synthetic construct
  source 1..20: mol_type = other RNA
aaatgtgctagtgcctaaat

SEQ ID NO: 113 - RNA, 20 nt, synthetic construct
  source 1..20: mol_type = other RNA
aaatgtgctagtgccgaaat

SEQ ID NO: 114 - RNA, 20 nt, synthetic construct
  source 1..20: mol_type = other RNA
aaatgtgctagtgcccaaat

SEQ ID NO: 115 - RNA, 20 nt, synthetic construct
  source 1..20: mol_type = other RNA
aaatgtgctagtgccataat

SEQ ID NO: 116 - RNA, 20 nt, synthetic construct
  source 1..20: mol_type = other RNA
aaatgtgctagtgccacaat

SEQ ID NO: 117 - RNA, 20 nt, synthetic construct
  source 1..20: mol_type = other RNA
aaatgtgctagtgccagaat

SEQ ID NO: 118 - RNA, 20 nt, synthetic construct
  source 1..20: mol_type = other RNA
aaatgtgctagtgccaatat

SEQ ID NO: 119 - RNA, 20 nt, synthetic construct
  source 1..20: mol_type = other RNA
aaatgtgctagtgccaacat

SEQ ID NO: 120 - RNA, 20 nt, synthetic construct
  source 1..20: mol_type = other RNA
aaatgtgctagtgccaagat

SEQ ID NO: 121 - RNA, 20 nt, synthetic construct
  source 1..20: mol_type = other RNA
aaatgtgctagtgccaaatt

SEQ ID NO: 122 - RNA, 20 nt, synthetic construct
  source 1..20: mol_type = other RNA
aaatgtgctagtgccaaact

SEQ ID NO: 123 - RNA, 20 nt, synthetic construct
  source 1..20: mol_type = other RNA
aaatgtgctagtgccaaagt

SEQ ID NO: 124 - RNA, 20 nt, synthetic construct
  source 1..20: mol_type = other RNA
aaatgtgctagtgccaaaaa

SEQ ID NO: 125 - RNA, 20 nt, synthetic construct
  source 1..20: mol_type = other RNA
aaatgtgctagtgccaaaac

SEQ ID NO: 126 - RNA, 20 nt, synthetic construct
  source 1..20: mol_type = other RNA
aaatgtgctagtgccaaaag

SEQ ID NO: 127 - RNA, 20 nt, synthetic construct
  source 1..20: mol_type = other RNA
aaatgtgctagttccaaaat

SEQ ID NO: 128 - RNA, 20 nt, synthetic construct
  source 1..20: mol_type = other RNA
aaatgtgctagagccaaaat

SEQ ID NO: 129 - RNA, 20 nt, synthetic construct
  source 1..20: mol_type = other RNA
aaatgtgctagttccaaaat

SEQ ID NO: 130 - RNA, 21 nt, synthetic construct
  source 1..21: mol_type = other RNA
caagcaaaaatgtgctagttc

SEQ ID NO: 131 - RNA, 21 nt, synthetic construct
  source 1..21: mol_type = other RNA
caagcaaaaatgtgctagtgt

SEQ ID NO: 132 - RNA, 21 nt, synthetic construct
  source 1..21: mol_type = other RNA
caagcaaaaatgtgctagagc

SEQ ID NO: 133 - RNA, 21 nt, synthetic construct
  source 1..21: mol_type = other RNA
tctccgctctgagcaatcacg

SEQ ID NO: 134 - RNA, 21 nt, synthetic construct
  source 1..21: mol_type = other RNA
gctagttccaaaatcggccaa

SEQ ID NO: 135 - RNA, 21 nt, synthetic construct
  source 1..21: mol_type = other RNA
gctagtgtcaaaatcggccaa

SEQ ID NO: 136 - RNA, 21 nt, synthetic construct
  source 1..21: mol_type = other RNA
gctagagccaaaatcggccaa

SEQ ID NO: 137 - RNA, 21 nt, synthetic construct
  source 1..21: mol_type = other RNA
ctctgagcaatcacgtgcagt

SEQ ID NO: 138 - RNA, 20 nt, synthetic construct
  source 1..20: mol_type = other RNA
agttccaaaatcggccaagc

SEQ ID NO: 139 - RNA, 20 nt, synthetic construct
  source 1..20: mol_type = other RNA
gctagttccaaaatcggcca

SEQ ID NO: 140 - RNA, 20 nt, synthetic construct
  source 1..20: mol_type = other RNA
aatgtgctagttccaaaatc

SEQ ID NO: 141 - RNA, 20 nt, synthetic construct
  source 1..20: mol_type = other RNA
gcttggccgattttggaact

SEQ ID NO: 142 - RNA, 20 nt, synthetic construct
  source 1..20: mol_type = other RNA
agtgtcaaaatcggccaagc

SEQ ID NO: 143 - RNA, 20 nt, synthetic construct
  source 1..20: mol_type = other RNA
gctagtgtcaaaatcggcca

SEQ ID NO: 144 - RNA, 20 nt, synthetic construct
  source 1..20: mol_type = other RNA
aatgtgctagtgtcaaaatc

SEQ ID NO: 145 - RNA, 20 nt, synthetic construct
  source 1..20: mol_type = other RNA
aaatgtgctagtgtcaaaat

SEQ ID NO: 146 - RNA, 20 nt, synthetic construct
  source 1..20: mol_type = other RNA
gcttggccgattttgacact

SEQ ID NO: 147 - RNA, 20 nt, synthetic construct
  source 1..20: mol_type = other RNA
agagccaaaatcggccaagc

SEQ ID NO: 148 - RNA, 20 nt, synthetic construct
  source 1..20: mol_type = other RNA
gctagagccaaaatcggcca

SEQ ID NO: 149 - RNA, 20 nt, synthetic construct
  source 1..20: mol_type = other RNA
aatgtgctagagccaaaatc

SEQ ID NO: 150 - RNA, 20 nt, synthetic construct
  source 1..20: mol_type = other RNA
aaatgtgctagagccaaaat

SEQ ID NO: 151 - RNA, 20 nt, synthetic construct
  source 1..20: mol_type = other RNA
gcttggccgattttggctct

SEQ ID NO: 152 - RNA, 20 nt, synthetic construct
  source 1..20: mol_type = other RNA
ctgcacgtgattgctcagag

SEQ ID NO: 153 - RNA, 20 nt, synthetic construct
  source 1..20: mol_type = other RNA
cactgcacgtgattgctcag

SEQ ID NO: 154 - RNA, 20 nt, synthetic construct
  source 1..20: mol_type = other RNA
ggcactgcacgtgattgctc

SEQ ID NO: 155 - RNA, 20 nt, synthetic construct
  source 1..20: mol_type = other RNA
atattggcactgcacgtgat

SEQ ID NO: 156 - RNA, 20 nt, synthetic construct
  source 1..20: mol_type = other RNA
tcccatattggcactgcacg

SEQ ID NO: 157 - RNA, 20 nt, synthetic construct
  source 1..20: mol_type = other RNA
ctccgctctgagcaatcacg

SEQ ID NO: 158 - RNA, 20 nt, synthetic construct
  source 1..20: mol_type = other RNA
cgctctgagcaatcacgtgc

SEQ ID NO: 159 - RNA, 20 nt, synthetic construct
  source 1..20: mol_type = other RNA
ctctgagcaatcacgtgcag

SEQ ID NO: 160 - RNA, 20 nt, synthetic construct
  source 1..20: mol_type = other RNA
atattggcactgcacgtgat

SEQ ID NO: 161 - RNA, 20 nt, synthetic construct
  source 1..20: mol_type = other RNA
atcacgtgcagtgccaatat

SEQ ID NO: 162 - RNA, 20 nt, synthetic construct
  source 1..20: mol_type = other RNA
tcacgtgcagtgccaatatg

SEQ ID NO: 163 - RNA, 20 nt, synthetic construct
  source 1..20: mol_type = other RNA
aatcacgtgcagtgccaata

SEQ ID NO: 164 - RNA, 20 nt, synthetic construct
  source 1..20: mol_type = other RNA
atcacgtgcagtgccaatat

SEQ ID NO: 165 - RNA, 20 nt, synthetic construct
  source 1..20: mol_type = other RNA
tcacgtgcagtgccaatatg

SEQ ID NO: 166 - RNA, 20 nt, synthetic construct
  source 1..20: mol_type = other RNA
ttttgacactagcacatttt

SEQ ID NO: 167 - RNA, 20 nt, synthetic construct
  source 1..20: mol_type = other RNA
gccatctgcttggccgattt

SEQ ID NO: 168 - RNA, 80 nt, synthetic construct
  source 1..80: mol_type = other RNA
gttttagtactctggaaacagaatctactaaaacaaggcaaaatgccgtgtttatctcgtcaacttgttggcgagatttt

SEQ ID NO: 169 - RNA, 88 nt, synthetic construct
  source 1..88: mol_type = other RNA
gttatagtactctgtaatgaaaattacagaatctactataacaaggcaaaatgccgtgtttatctcgtcaacttgttggcgagatttt

SEQ ID NO: 170 - RNA, 82 nt, synthetic construct
  source 1..82: mol_type = other RNA
gttttagagctagaaatagcaagttaaaataaggctagtccgttatcaacttgaaaaagtggcaccgagtcggtgctttttt

SEQ ID NO: 171 - RNA, 92 nt, synthetic construct
  source 1..92: mol_type = other RNA
gtttcagagctatgctggaaacagcatagcaagttgaaataaggctagtccgttatcaacttgaaaaagtggcaccgagtcggtgctttttt

SEQ ID NO: 172 - RNA, 78 nt, synthetic construct
  source 1..78: mol_type = other RNA
tggccgattttggcactagcacatttttgcttgtgtctctccgctctgagcaatcatgtgcagtgccaatatgggaaa

SEQ ID NO: 173 - protein, 7 aa, synthetic construct
PKKKRKV

SEQ ID NO: 174 - protein, 7 aa, synthetic construct
PKKKRRV

SEQ ID NO: 175 - protein, 16 aa, synthetic construct
KRPAATKKAGQAKKKK

SEQ ID NO: 176 - RNA, 39 nt, synthetic construct
  source 1..39: mol_type = other RNA
cttggccgattttggaactagcacatttttgcttgtgtc

SEQ ID NO: 177 - RNA, 40 nt, synthetic construct
  source 1..40: mol_type = other RNA
gaaccggctaaaaccttgatcgtgtaaaaacgaacacagg

SEQ ID NO: 178 - RNA, 23 nt, synthetic construct
  source 1..23: mol_type = other RNA
  modified_base 7: mod_base = OTHER; note = Thymine
  modified_base 21: mod_base = OTHER; note = Thymine
aaaatgtgctagttccaaaatcg

SEQ ID NO: 179 - RNA, 24 nt, synthetic construct
  source 1..24: mol_type = other RNA
  modified_base 21: mod_base = OTHER; note = Thymine
aaaatgtgctagttccaaaatcgg

SEQ ID NO: 180 - RNA, 32 nt, synthetic construct
  source 1..32: mol_type = other RNA
ccgattttggaactagcacatttttgcttgtg

SEQ ID NO: 181 - RNA, 32 nt, synthetic construct
  source 1..32: mol_type = other RNA
ccgattttggcactagcacatttttgcttgtg

SEQ ID NO: 182 - RNA, 26 nt, synthetic construct
  source 1..26: mol_type = other RNA
ccgattttggaacatttttgcttgtg

SEQ ID NO: 183 - RNA, 26 nt, synthetic construct
  source 1..26: mol_type = other RNA
ccgattttggcacatttttgcttgtg

SEQ ID NO: 184 - RNA, 30 nt, synthetic construct
  source 1..30: mol_type = other RNA
ccgattttggctagcacatttttgcttgtg

SEQ ID NO: 185 - RNA, 32 nt, synthetic construct
  source 1..32: mol_type = other RNA
ccgattttggaaactagcacatttttgcttgt

SEQ ID NO: 186 - RNA, 16 nt, synthetic construct
  source 1..16: mol_type = other RNA
ccgatttttgcttgtg

SEQ ID NO: 187 - RNA, 28 nt, synthetic construct
  source 1..28: mol_type = other RNA
ccgattttctagcacatttttgcttgtg

SEQ ID NO: 188 - RNA, 15 nt, synthetic construct
  source 1..15: mol_type = other RNA
ccgattttgcttgtg

SEQ ID NO: 189 - RNA, 16 nt, synthetic construct
  source 1..16: mol_type = other RNA
cacatttttgcttgtg

SEQ ID NO: 190 - RNA, 29 nt, synthetic construct
  source 1..29: mol_type = other RNA
ccgattttggaagcacatttttgcttgtg

SEQ ID NO: 191 - RNA, 26 nt, synthetic construct
  source 1..26: mol_type = other RNA
ccgattctagcacatttttgcttgtg

SEQ ID NO: 192 - RNA, 31 nt, synthetic construct
  source 1..31: mol_type = other RNA
ccgattttggactagcacatttttgcttgtg

SEQ ID NO: 193 - RNA, 29 nt, synthetic construct
  source 1..29: mol_type = other RNA
ccgattttgctagcacatttttgcttgtg

SEQ ID NO: 194 - RNA, 27 nt, synthetic construct
  source 1..27: mol_type = other RNA
ccgatttctagcacatttttgcttgtg

SEQ ID NO: 195 - RNA, 31 nt, synthetic construct
  source 1..31: mol_type = other RNA
ccgattttggaatagcacatttttgcttgtg

SEQ ID NO: 196 - RNA, 24 nt, synthetic construct
  source 1..24: mol_type = other RNA
ccgattttggaatttttgcttgtg

SEQ ID NO: 197 - RNA, 28 nt, synthetic construct
  source 1..28: mol_type = other RNA
ccgattttggaacacatttttgcttgtg

SEQ ID NO: 198 - RNA, 20 nt, synthetic construct
  source 1..20: mol_type = other RNA
ctagcacatttttgcttgtg

SEQ ID NO: 199 - RNA, 28 nt, synthetic construct
  source 1..28: mol_type = other RNA
ccgattttggaacacatttttgcttgtg

SEQ ID NO: 200 - RNA, 32 nt, synthetic construct; identical to SEQ ID NO: 181
  source 1..32: mol_type = other RNA

SEQ ID NO: 201 - RNA, 32 nt, synthetic construct
  source 1..32: mol_type = other RNA
ccgattgtggcactagcacattgttgcttgtg

SEQ ID NO: 202 - RNA, 31 nt, synthetic construct
  source 1..31: mol_type = other RNA
ccgatttggcactagcacatttttgcttgtg

SEQ ID NO: 203 - RNA, 32 nt, synthetic construct
  source 1..32: mol_type = other RNA
ccgattttggcactagctcatttttgcttgtg

SEQ ID NO: 204 - RNA, 33 nt, synthetic construct
  source 1..33: mol_type = other RNA
ccgatttttgcactaagcacatttttgcttgtg

SEQ ID NO: 205 - RNA, 32 nt, synthetic construct
  source 1..32: mol_type = other RNA
ccgtttttggcactagcacatttttgcttgtg

SEQ ID NO: 206 - RNA, 101 nt, synthetic construct
  source 1..101: mol_type = other RNA
nnnnnnnnnnnnnnnnnnnnngttttagtactctggaaacagaatctactaaaacaaggcaaaatgccgtgtttatctcgtcaacttgttggcgagatttt

SEQ ID NO: 207 - RNA, 109 nt, synthetic construct
  source 1..109: mol_type = other RNA
nnnnnnnnnnnnnnnnnnnnngttatagtactctgtaatgaaaattacagaatctactataacaaggcaaaatgccgtgtttatctcgtcaacttgttggcgagatttt

SEQ ID NO: 208 - RNA, 40 nt, synthetic construct
  source 1..40: mol_type = other RNA
ctgcttggccgattttggaactagcacatttttgcttgtg

SEQ ID NO: 209 - RNA, 40 nt, synthetic construct
  source 1..40: mol_type = other RNA
ctgcttggccgattttggcactagcacatttttgcttgtg

SEQ ID NO: 210 - RNA, 40 nt, synthetic construct
  source 1..40: mol_type = other RNA
ctgcttggccgattttggaaactagcacatttttgcttgt

SEQ ID NO: 211 - RNA, 29 nt, synthetic construct
  source 1..29: mol_type = other RNA
ctgcttggccgattttatttttgcttgtg

SEQ ID NO: 212 - RNA, 34 nt, synthetic construct
  source 1..34: mol_type = other RNA
ctgcttggccgattttggaacatttttgcttgtg

SEQ ID NO: 213 - RNA, 38 nt, synthetic construct
  source 1..38: mol_type = other RNA
ctgcttggccgattttggctagcacatttttgcttgtg

SEQ ID NO: 214 - RNA, 40 nt, synthetic construct
  source 1..40: mol_type = other RNA
ctgcttggccgattttggaactagcacatttttgcttttg

SEQ ID NO: 215 - RNA, 40 nt, synthetic construct
  source 1..40: mol_type = other RNA
ctgcttggccgattttggaactagcacatttttgcttgtt

SEQ ID NO: 216 - RNA, 40 nt, synthetic construct; identical to SEQ ID NO: 209
  source 1..40: mol_type = other RNA

SEQ ID NO: 217 - RNA, 39 nt, synthetic construct
  source 1..39: mol_type = other RNA
ctgcttggccgattttggcactagcacatttttgcttgt

SEQ ID NO: 218 - RNA, 38 nt, synthetic construct
  source 1..38: mol_type = other RNA
gcttggccgattttggaactagcacatttttgcttgtg

SEQ ID NO: 219 - RNA, 38 nt, synthetic construct
  source 1..38: mol_type = other RNA
gcttggccgattttggcactagcacatttttgcttgtg

SEQ ID NO: 220 - RNA, 36 nt, synthetic construct
  source 1..36: mol_type = other RNA
gcttggccgattttggctagcacatttttgcttgtg

SEQ ID NO: 221 - RNA, 34 nt, synthetic construct
  source 1..34: mol_type = other RNA
gcttggccgattttctagcacatttttgcttgtg

SEQ ID NO: 222 - RNA, 38 nt, synthetic construct
  source 1..38: mol_type = other RNA
gcttggccgattttgacactagcacatttttgcttgtg

SEQ ID NO: 223 - RNA, 38 nt, synthetic construct
  source 1..38: mol_type = other RNA
gcttggccgattttgaaactagcacatttttgcttgtg

SEQ ID NO: 224 - RNA, 38 nt, synthetic construct
  source 1..38: mol_type = other RNA
gcttggccgattttggaaactagcacatttttgcttgt

SEQ ID NO: 225 - RNA, 37 nt, synthetic construct
  source 1..37: mol_type = other RNA
gcttggccgattttggaatagcacatttttgcttgtg

SEQ ID NO: 226 - RNA, 38 nt, synthetic construct
  source 1..38: mol_type = other RNA
gcttggccgattttggaaaactagcacatttttgcttg

SEQ ID NO: 227 - RNA, 36 nt, synthetic construct
  source 1..36: mol_type = other RNA
gcttggccgattttggaaagcacatttttgcttgtg

SEQ ID NO: 228 - RNA, 35 nt, synthetic construct
  source 1..35: mol_type = other RNA
gcttggccgattttgctagcacatttttgcttgtg

SEQ ID NO: 229 - RNA, 37 nt, synthetic construct
  source 1..37: mol_type = other RNA
gcttggccgattttggactagcacatttttgcttgtg

SEQ ID NO: 230 - RNA, 35 nt, synthetic construct
  source 1..35: mol_type = other RNA
gcttggccgattttggaagcacatttttgcttgtg

SEQ ID NO: 231 - RNA, 33 nt, synthetic construct
  source 1..33: mol_type = other RNA
gcttggccgatttctagcacatttttgcttgtg

SEQ ID NO: 232 - RNA, 32 nt, synthetic construct
  source 1..32: mol_type = other RNA
gcttggccgattctagcacatttttgcttgtg

SEQ ID NO: 233 - RNA, 40 nt, synthetic construct
  source 1..40: mol_type = other RNA
cttggccgattttggcactagcacatttttgcttgtgtcc

SEQ ID NO: 234 - RNA, 40 nt, synthetic construct
  source 1..40: mol_type = other RNA
gaaccggctaaaaccgtgatcgtgtaaaaacgaacacagg

SEQ ID NO: 235 - RNA, 23 nt, synthetic construct
  source 1..23: mol_type = other RNA
aaatgtgctagttccaaaatcgg

SEQ ID NO: 236 - RNA, 23 nt, synthetic construct
  source 1..23: mol_type = other RNA
aaatgtgctagtgtcaaaatcgg

SEQ ID NO: 237 - RNA, 23 nt, synthetic construct
  source 1..23: mol_type = other RNA
aaatgtgctagagccaaaatcgg

SEQ ID NO: 238 - RNA, 111 nt, synthetic construct
  source 1..111: mol_type = other RNA
nnnnnnnnnnnnnnnnnnnngtttcagagctatgctggaaacagcatagcaagttgaaataaggctagtccgttatcaacttgaaaagtggcaccgagtcggtgctttttt

SEQ ID NO: 239 - RNA, 40 nt, synthetic construct
  source 1..40: mol_type = other RNA
tgccatctgcttggccgattttggctctagcacatttttg

SEQ ID NO: 240 - RNA, 40 nt, synthetic construct
  source 1..40: mol_type = other RNA
tgccatctgcttggccgatttttggctctagcacattttt

SEQ ID NO: 241 - RNA, 39 nt, synthetic construct
  source 1..39: mol_type = other RNA
tgccatctgcttggccgatttggctctagcacatttttg

SEQ ID NO: 242 - RNA, 30 nt, synthetic construct
  source 1..30: mol_type = other RNA
tgccatctgcttggctctagcacatttttg

SEQ ID NO: 243 - RNA, 38 nt, synthetic construct
  source 1..38: mol_type = other RNA
tgccatctgcttggccgattggctctagcacatttttg

SEQ ID NO: 244 - RNA, 24 nt, synthetic construct
  source 1..24: mol_type = other RNA
tgccatctgcttggccgatttttg

SEQ ID NO: 245 - RNA, 40 nt, synthetic construct
  source 1..40: mol_type = other RNA
tgccatctgcttggccgattttttggctctagcacatttt

SEQ ID NO: 246 - RNA, 37 nt, synthetic construct
  source 1..37: mol_type = other RNA
tgccatctgcttggccgattgctctagcacatttttg

SEQ ID NO: 247 - RNA, 34 nt, synthetic construct
  source 1..34: mol_type = other RNA
tgccatctgcttggccgattctagcacatttttg

SEQ ID NO: 248 - RNA, 23 nt, synthetic construct
  source 1..23: mol_type = other RNA
tgccatctgcttggccgattttg

SEQ ID NO: 249 - RNA, 25 nt, synthetic construct
  source 1..25: mol_type = other RNA
tgccatctgctctagcacatttttg

SEQ ID NO: 250 - RNA, 35 nt, synthetic construct
  source 1..35: mol_type = other RNA
tgccatctgcttggccgatttctagcacatttttg

SEQ ID NO: 251 - RNA, 33 nt, synthetic construct
  source 1..33: mol_type = other RNA
tgccatctgcttggccgatttagcacatttttg

SEQ ID NO: 252 - RNA, 24 nt, synthetic construct
  source 1..24: mol_type = other RNA
tgccatctgcttggcacatttttg

SEQ ID NO: 253 - RNA, 32 nt, synthetic construct
  source 1..32: mol_type = other RNA
tgccatctgcttggccgattagcacatttttg

SEQ ID NO: 254 - RNA, 20 nt, synthetic construct
  source 1..20: mol_type = other RNA
tgccatctgcttggccgatt

SEQ ID NO: 255 - RNA, 29 nt, synthetic construct
  source 1..29: mol_type = other RNA
tgccatctgcttggccgagcacatttttg

SEQ ID NO: 256 - RNA, 40 nt, synthetic construct
  source 1..40: mol_type = other RNA
tgccatctgcttggccgatttttgacactagcacattttt

SEQ ID NO: 257 - RNA, 30 nt, synthetic construct
  source 1..30: mol_type = other RNA
tgccatctgcttggccgattcacatttttg

SEQ ID NO: 258 - RNA, 13 nt, synthetic construct
  source 1..13: mol_type = other RNA
tgccatctgcttg

SEQ ID NO: 259 - RNA, 40 nt, synthetic construct
  source 1..40: mol_type = other RNA
tgccatctgcttggccgattttgacactagcacatttttg

SEQ ID NO: 260 - RNA, 39 nt, synthetic construct
  source 1..39: mol_type = other RNA
gccatctgcttggccgattttgttggctctagcacattt

SEQ ID NO: 261 - RNA, 25 nt, synthetic construct
  source 1..25: mol_type = other RNA
tgccatctgcttggccgattttttg

SEQ ID NO: 262 - RNA, 40 nt, synthetic construct
  source 1..40: mol_type = other RNA
tgccatctgcttggccgattattggctctagcacattttt

SEQ ID NO: 263 - RNA, 23 nt, synthetic construct
  source 1..23: mol_type = other RNA
tgccatctgctagcacatttttg

SEQ ID NO: 264 - RNA, 29 nt, synthetic construct
  source 1..29: mol_type = other RNA
tgccatctgcttggccgattacatttttg

SEQ ID NO: 265 - RNA, 34 nt, synthetic construct
  source 1..34: mol_type = other RNA
tgccatctgcttggccgattttagcacatttttg

SEQ ID NO: 266 - RNA, 22 nt, synthetic construct
  source 1..22: mol_type = other RNA
tgccatctgcttggccgatttg

SEQ ID NO: 267 - RNA, 37 nt, synthetic construct
  source 1..37: mol_type = other RNA
tgccatctgcttggccgatggctctagcacatttttg

SEQ ID NO: 268 - RNA, 32 nt, synthetic construct
  source 1..32: mol_type = other RNA
tgccatctgcttggccgatttgcacatttttg

SEQ ID NO: 269 - RNA, 39 nt, synthetic construct
  source 1..39: mol_type = other RNA
tgccatctgcttggccgatttgacactagcacatttttg

SEQ ID NO: 270 - RNA, 38 nt, synthetic construct
  source 1..38: mol_type = other RNA
tgccatctgcttggccgatttgctctagcacatttttg

SEQ ID NO: 271 - RNA, 40 nt, synthetic construct
  source 1..40: mol_type = other RNA
tgccatctgcttggccgattgttggctctagcacattttt

SEQ ID NO: 272 - RNA, 36 nt, synthetic construct
  source 1..36: mol_type = other RNA
tgccatctgcttggccgattctctagcacatttttg

SEQ ID NO: 273 - RNA, 31 nt, synthetic construct
  source 1..31: mol_type = other RNA
tgccatctgctttggctctagcacatttttg

SEQ ID NO: 274 - RNA, 20 nt, synthetic construct
  source 1..20: mol_type = other RNA
tgccatctagcacatttttg

SEQ ID NO: 275 - RNA, 40 nt, synthetic construct
  source 1..40: mol_type = other RNA
tgccatctgcttggccgattttggcactagcacatttttg

SEQ ID NO: 276 - RNA, 40 nt, synthetic construct; identical to SEQ ID NO: 239
  source 1..40: mol_type = other RNA

SEQ ID NO: 277 - RNA, 39 nt, synthetic construct; identical to SEQ ID NO: 241
  source 1..39: mol_type = other RNA

SEQ ID NO: 278 - RNA, 40 nt, synthetic construct; identical to SEQ ID NO: 240
  source 1..40: mol_type = other RNA

SEQ ID NO: 279 - RNA, 263 nt, synthetic construct
  source 1..263: mol_type = other RNA
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacac

SEQ ID NO: 280 - RNA, 46 nt, synthetic construct
  source 1..46: mol_type = other RNA
ctcaactggtgtcgtggagtcggcaattcagttgagcaaaaatgtg

SEQ ID NO: 281 - RNA, 22 nt, synthetic construct
  source 1..22: mol_type = other RNA
tcggcaggtttggaactagcac

SEQ ID NO: 282 - RNA, 18 nt, synthetic construct
  source 1..18: mol_type = other RNA
ctcaactggtgtcgtgga

SEQ ID NO: 283 - RNA, 23 nt, synthetic construct
  source 1..23: mol_type = other RNA
aaatgtgctagttccaaaatcgg

SEQ ID NO: 284 - RNA, 29 nt, synthetic construct
  source 1..29: mol_type = other RNA
  modified_base 29: mod_base = OTHER; note = Thymine
aaatgtgctagttccaaaatcggcaaaat

(19) United States
(12) Patent Application Publication
(10) Pub. No.: US 2025/0325700 A1
(43) Pub. Date: Oct. 23, 2025

(54) COMPOSITIONS AND METHODS FOR HEARING LOSS

(71) Applicant: Massachusetts Eye and Ear Infirmary, Boston, MA (US)

(72) Inventors: Zheng-Yi Chen, Chestnut Hill, MA (US); Wenliang Zhu, Malden, MA (US); Wan Du, Cambridge, MA (US)

(21) Appl. No.: 18/866,817

(22) PCT Filed: May 22, 2023
(86) PCT No.: PCT/US2023/023052

§ 371 Date: Nov. 18, 2024

Related U.S. Application Data
(60) Provisional application No. 63/344,596, filed on May 22, 2022.

(51) Int. Cl. A61K 48/00; A61P 27/16; C12N 9/22; C12N 15/11; C12N 15/86; C12N 15/90

(52) U.S. Cl. CPC A61K 48/005; A61P 27/16; C12N 9/226; C12N 15/11; C12N 15/86; C12N 15/907; C12N 2310/20; C12N 2750/14143

(57) ABSTRACT

Methods and compositions for use in treating subjects with non-syndromic progressive hearing loss caused by mutations of the miR96 by disruption of the mutant allele, and methods of use thereof, as well as genetically modified animals and cells.

### CROSS-REFERENCE TO RELATED APPLICATIONS

This application claims the benefit of U.S. Provisional Application Ser. No. 63/344,596, filed on May 22, 2022, the contents of which are incorporated herein by reference in their entirety.

### FEDERALLY SPONSORED RESEARCH OR DEVELOPMENT

This invention was made with government support under grant number DC016875 awarded by The National Institutes of Health. The government has certain rights in the invention.

### TECHNICAL FIELD

Described herein are compositions for use in treating subjects with miR96-associated non-syndromic progressive hearing loss caused by mutations of the miR96 locus by disruption of the mutant allele, and methods of use thereof, as well as genetically modified animals and cells.

### BACKGROUND

Hearing loss is a multi-factorial condition affecting a significant portion of the global population. Genetic mutations causing hearing loss account for more than 50% of all congenital sensorineural hearing loss (SNHL), yet few treatments are available to slow or reverse SNHL caused by genetic mutations.

MiR96 is a sensory organ-specific miRNA involved in mammalian cochlea development and hearing maintenance. MiR96 regulates the progression of differentiation in cochlear inner and outer hair cells. In humans, heterozygous point mutations in the seed region of the miRNA result in progressive hearing loss with autosomal dominant inheritance pattern, which indicate gain-of-function effects of the mutations. In humans, point mutations in the miR96 locus cause non-syndromic progressive hearing loss, beginning from children to adults, which offers an opportunity for genetic interference even for adult patients. At least two mutations of the locus encoding miR96, +13 G>A and +14 C>A, are associated with SNHL in humans.

Clustered regularly interspaced short palindromic repeats (CRISPR) mediated genome editing technology shows great promise as tools for treating various genetic diseases, including SNHL. However, previous treatments have been studied in neonatal animal models, where the cochleae are not mature, temporally equivalent to the human cochlea before 26 weeks of gestational age, while newborn human inner ears are fully developed. The cochlea undergoes significant developmental changes from neonatal to adult stages including changes in size, structure, and function. As such, genome editing in mature inner car cells is a challenge. Further, multiple mutant alleles of miR96 are associated with SNHL in humans. Therefore, there exists a need for genome editing compositions and methods for the treatment of SNHL by disrupting the multiple mutant alleles of miR96 associated with disease.

### SUMMARY

The compositions and methods provided herein are based, at least in part, on the discovery that the genome editing systems disclosed herein can efficiently and specifically disrupt disease-associated mutant alleles of the miR96 locus in inner and outer hair cells in mature mammalian cochlea. The compositions and methods disclosed herein provide for efficient delivery of a genome editing complex into hair cells of mature cochlea. Further, the compositions and methods disclosed herein can promote survival of hair cells in mature mammalian cochlea and ameliorate bearing loss in a subject. Further still, the compositions and methods disclosed herein can be used to treat subjects harboring different disease-associated alleles by administration of the same single composition.

In a first aspect, the disclosure provides compositions including one or more nucleic acids including a sequence encoding an RNA-guided nuclease and a sequence encoding one or more single guide RNAs (sgRNAs), wherein the target sequence of the one or more sgRNAs includes a mutation of the miR-96 locus selected from the group consisting of +14 C>A, +13 G>A, and +15 A>T relative to SEQ ID NO: 172. In some embodiments, the target sequence of the one or more sgRNAs comprises any one of SEQ ID NOs 1-167. In some embodiments, the RNA-guided nuclease is a Cas9 nuclease. In some embodiments, the Cas9 nuclease is selected from the group consisting of spCas9 or variant thereof, saCas9 or variant thereof, scCas9++, LZ3 Cas9, KKH-saCas9 and sauriCas9. In some embodiments, the one or more sgRNAs each further includes a sequence selected from any one of SEQ ID NOs. 168-171. In some embodiments, the RNA-guided nuclease includes one or more nuclear localization signals. In some embodiments, the one or more nuclear localization signals includes a C-terminal nuclear localization signal and/or an N-terminal nuclear localization signal. In some embodiments, the sequence encoding the RNA-guided nuclease includes a polyadenylation signal.

In some embodiments, the one or more nucleic acids is a viral delivery vector. In some embodiments, the viral delivery vector is an adenovirus vector, an adeno-associated virus (AAV) vector, or a lentivirus vector. In some embodiments, a first nucleic acid comprises the sequence encoding the RNA-guided nuclease and a second nucleic acid comprises the sequence encoding the one or more sgRNAs. In some embodiments, the second nucleic acid includes: (i) a first sgRNA that targets the +14 C>A mutation of the miR-96 locus; (ii) a second sgRNA that targets the +13 G>A mutation of the miR-96 locus; and (iii) a third sgRNA that targets the +15 A>T mutation of the miR-96 locus. In some embodiments, the first sgRNA includes SEQ ID NOs: 129 and 171, the second sgRNA includes SEQ ID NOs: 127 and 171, and the third sgRNA includes SEQ ID NOs: 128 and 171.

In some embodiments, the composition is for use in therapy. In some embodiments, the composition is for use in preparation of a medicament. In some embodiments, the composition is for use in a method of treating a subject who has non-syndromic progressive hearing loss. In some embodiments, the AAV vector is delivered to the inner ear of a subject by injection, optionally through the round window. In another aspect, the disclosure provides compositions including a ribonucleoprotein (RNP) complex including an RNA-guided nuclease and an sgRNA, wherein the target sequence of the sgRNA is any one of SEQ ID NOs 1-167. In some embodiments, the Cas9 nuclease is selected from the group consisting of spCas9, scCas9++, LZ3 Cas9, KKH-saCas9 and sauriCas9. In some embodiments, the sgRNA includes: (i) SEQ ID NOs: 129 and 171; (ii) SEQ ID NOs: 127 and 171; or (iii) SEQ ID NOs: 128 and 171.

In another aspect, the disclosure provides methods of disrupting a mutant allele of the miR-96 locus in a cell, the mutant allele being selected from the group consisting of +14 C>A, +13 G>A, and +15 A>T relative to SEQ ID NO: 172, further including contacting the cell with the compositions disclosed herein. In some embodiments, disrupting the mutant allele is effected using a sgRNA having a target sequence of any one of SEQ ID NOs 1-167. In some embodiments, the cell is in or from a subject who has non-syndromic progressive hearing loss. In some embodiments, the cell is a cell of the inner ear of the subject. In some embodiments, the cell is an outer hair cell.

In another aspect, the disclosure features methods of treating progressive non-syndromic hearing loss in a patient in need thereof, the method comprising administering to the patient the compositions disclosed herein. In some embodiments, the patient harbors a mutation of the miR-96 locus selected from the group consisting of +14 C>A, +13 G>A, and +15 A>T relative to SEQ ID NO: 172.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a schematic of the the miR-96 locus with the +14 C>A Dmdo mutation indicated by an asterisk, and several of the sgRNAs disclosed herein.

FIG. 1B is a schematic overview of plasmid structure for different CRISPR systems disclosed herein. Various combinations of Cas9 enzymes and sgRNAs are indicated.

FIG. 1C is a schematic overview of preparation of primary fibroblasts for editing the miR-96 locus in the Dmdo mouse model.

FIG. 1D is a bar chart of the indel frequency in miR-96^{Dmdo/+} primary fibroblasts after genome editing. Error bars represent standard error of the mean.

FIG. 1E is a schematic of representative next generation sequencing (NGS) results from KKH-saCas9/sgRNA-3 edited miR-96^{Dmdo/+} fibroblasts.

FIG. 1F is a schematic of representative next generation sequencing (NGS) results from KKH-saCas9/sgRNA-3 edited miR-96^{+/+} fibroblasts.

FIG. 2A is a Schematic overview of establishment of miR-96 14 C-to-A HEK-293T cell line.

FIG. 2B is a bar chart showing the editing efficiency of miR-96 Dmdo mutation loci and wild-type loci using spCas9/sgRNA-1 and KKH-saCas9/sgRNA-4 in mouse and human cells. Error bars represent standard error of the mean.

FIG. 2C is a plot of the indel profile for the spCas9/sgRNA-1 combination.

FIG. 2D is a plot of the indel profile for the KKH-saCas9/sgRNA-4 combination.

FIG. 3A is a schematic of the design of the optimized AAV structure of KKH-saCas9/sgRNA vector.

FIG. 3B is a schematic of the sequence of the original KKH-saCas9 sgRNA.

FIG. 3C is a schematic of the sequence of the optimized KKH-saCas9 sgRNA, with bold letters indicating changes relative to the original sequence.

FIG. 3D is a representative fluorescent image of tdTomato-positive mouse fibroblasts indicating editing efficiency for optimized KKH-saCas9/sgRNA relative to the original sequence.

FIG. 3E is a plot of quantification of the fluorescent images represented in FIG. 3D, indicating editing efficiency for optimized KKH-saCas9/sgRNA relative to the original sequence. Error bars represent standard error of the mean.

FIG. 3F is a representative fluorescent image of EGFP-negative human HEK cells indicating editing efficiency for optimized KKH-saCas9/sgRNA relative to the original sequence.

FIG. 3G is a plot of quantification of the fluorescent images represented in FIG. 3F, indicating editing efficiency for optimized KKH-saCas9/sgRNA relative to the original sequence. Error bars represent standard error of the mean.

FIG. 4A is a schematic overview of AAV2 construction, production, and injection in adult mice cochlea.

FIG. 4B is a schematic of the experimental overview for in vivo mouse studies.

FIG. 4C is a Representative indels analysis from the whole cochlea AAV2-KKH-saCas9/sgRNA-4 injected mice 2 month after the injection.

FIG. 5 is a plot of ABR thresholds of 3.5 months after AAV2 injection, injected with AAV2 ears (square datapoints) and uninjected ears (circle datapoints).

FIG. 6A is a schematic overview of the experimental protocol of hair cell isolation, cell lysis and deep sequencing.

FIG. 6B is a representative NGS result of isolated hair cells lysis from AAV2-KKH-saCas9-sgRNA-4 injected cochlea.

FIG. 6C is a plot of quantification of miR-96 s1334 allele-specific indel frequency in the NGS results of AAV2-KKH-saCas9-sgRNA-4 injected hair cells lysis and cochlea samples (n=9). Each dot represents a unique sequencing reaction from three cochleae combination. Error bars represent standard deviation.

FIG. 6D is a series of pie charts of percentage of miR-96 wild-type allele reads, s1334 reads, and indel-containing reads in the NGS results from AAV2-KKH-saCas9-sgRNA-4 injected hair cells lysis from three independent experiments.

FIG. 7A is a schematic of sequence information of the human miR-96 locus and the sgRNAs designed to target three different disease-associated mutations. Arrows indicate the mutation locations. The protospacer adjacent motifs (PAMs) nucleotides are indicated by rectangles.

FIG. 7B is a schematic overview of dual AAV constructions. One contains “U1a-spCas9-polyA” cassette, the other contains multiple U6-sgRNA cassettes.

FIG. 7C is a schematic of the sequence of the optimized spCas9 sgRNA. Bold letters indicate changes relative to the original sequence.

FIG. 7D is a schematic of representative NGS results from spCas9/sgmiR96-master edited HEK-miR96 mutant (15 A-to-T) cells.

FIG. 7E is a schematic of representative NGS results from spCas9/sgmiR96-master edited HEK293T wild type cells.

FIG. 7F is a plot of indel profiles from spCas9/sgmiR96-master edited HEK-miR96 (15 A-to-T) cells. Indel size is represented on the x-axis and percentage of sequencing reads containing an indel is represented on the y-axis. For indel size (x-axis), negative numbers represent deletions and positive numbers represent insertions.

FIG. 7G is a plot of indel profiles from spCas9/sgmiR96-master edited HEK-miR96 (15 A-to-T) cells. Reference amplicon position is represented on the x-axis and percentage of sequencing reads containing an indel is represented on the y-axis.

FIG. 7H is a bar chart of the indel frequency in HEK-miR96 cells bearing the 13 G-to-A, 14 C-to-A, and 15 A-to-T mutations and wild-type HEK293T cells after genome editing using spCas9/sgmiR96-master. Error bar represents standard deviation.

FIG. 7I is a plot of off-target analysis in spCas9/sgmiR96-master edited HEK-miR96 (15 A-to-T) cells. Genomic DNA was pooled from three independent biological replicates for sequencing on one occasion.

FIG. 8A is a representative confocal microscopy whole mount image of AAV2-KKH-saCas9-sgRNA-4 injected miR-96s^{1334/+} mouse cochlea, featuring the apex. Cochlea was stained with MYO7A.

FIG. 8B is a representative confocal microscopy whole mount image uninjected negative control miR-96s^{1334/+} mouse cochlea, featuring the apex. Cochlea was stained with MYO7A. Degeneration of hair cells is indicated by asterisks.

FIG. 8C is a representative confocal microscopy whole mount image of AAV2-KKH-saCas9-sgRNA-4 injected miR-96s^{1334/+} mouse cochlea, featuring the middle of the cochlea. Cochlea was stained with MYO7A.

FIG. 8D is a representative confocal microscopy whole mount image uninjected negative control miR-96s^{1334/+} mouse cochlea, featuring the middle of the cochlea. Cochlea was stained with MYO7A. Degeneration of hair cells is indicated by asterisks.

FIG. 8E is a plot of the number of outer hair cells (OHCs) per 100 μm section for three untreated and three AAV2-KKH-saCas9-sgRNA-4 treated miR-96s^{1334/+} cochleae from three mice at 10 weeks after treatment. Values and error bars reflect the mean±standard deviation.

FIG. 8F is a plot of the number of inner hair cells (IHCs) per 100 μm section for three untreated and three AAV2-KKH-saCas9-sgRNA-4 treated miR-96s^{1334/+} cochleae from three mice at 10 weeks after treatment. Values and error bars reflect the mean±standard deviation.

FIG. 8G is a representative scanning electron microscopy (SEM) image of AAV2-KKH-saCas9-sgRNA-4 injected miR-96^{s1334/+} outer hair cell bundles at apex region.

FIG. 8H is a representative scanning electron microscopy (SEM) image of uninjected negative control miR-96^{s1334/+} outer hair cell bundles at apex region. Degenerated stereocilia are indicated by asterisks.

FIG. 8I is a representative high-magnfication scanning electron microscopy (SEM) image of AAV2-KKH-saCas9-sgRNA-4 injected miR-96^{s1334/+} outer hair cell bundles at apex region.

FIG. 8J is a representative high-magnification scanning electron microscopy (SEM) image of uninjected negative control miR-96^{s1334/+} outer hair cell bundles at apex region. Degenerated stereocilia are indicated by asterisks.

FIG. 8K is a representative scanning electron microscopy (SEM) image of AAV2-KKH-saCas9-sgRNA-4 injected miR-96^{s1334/+} inner hair cell bundles at apex region.

FIG. 8L is a representative scanning electron microscopy (SEM) image of uninjected miR-96^{s1334/+} inner hair cell bundles at apex region.

FIG. 8M is a representative high-magnification scanning electron microscopy (SEM) image of AAV2-KKH-saCas9-sgRNA-4 injected miR-96^{s1334/+} inner hair cell bundles at apex region.

FIG. 8N is a representative high-magnification scanning electron microscopy (SEM) image of uninjected miR-96^{s1334/+} inner hair cell bundles at apex region.

### DETAILED DESCRIPTION

### miR96-Associated Hearing Loss

MicroRNAs (miRNAs) bind to complementary sites in their target mRNAs to mediate post-transcriptional repression with the specificity of target recognition being dependent on the miRNA seed region. Impaired miRNA target binding resulting from single nucleotide variants within mRNA target sites, or within the seed region of the miRNA itself, have been shown to lead to pathologies associated with dysregulated gene expression.

MiR96 is a microRNA encoded at human genome cytogenetic location 7q32.2, Genomic coordinates (GRCh38): 7:129,774,692-129,774,769. The DNA sequence encoding miR96 is provided below: TGGCCGATTTTGGCACTAGCACATTTTTGCTTGTGTCTCTCCGCTCTGAGCAATCAT GTGCAGTGCCAATATGGGAAA (SEQ ID NO: 172) MiR-96 is expressed in developing cochlear hair and is thought to directly or indirectly affect the expression of a large number of downstream genes implicated in cochlear function, development, and survival. In humans, at least two single-nucleotide mutations located within the seed region of miR96 are associated with autosomal dominant progressive nonsyndromic hearing loss: +13 G>A and +14 C>A relative to SEQ ID NO: 172.

### RNA-Guided Nucleases/Cas9

Various RNA-guided nucleases can be used in the present methods, e.g., as described in WO 2018/026976. This approach can use different CRISPR proteins and their corresponding gRNAs, including Streptococcus pyogenes Cas9 (SpCas9) and engineered SpCas9 variants, Staphylococcus aureus Cas9 (SaCas9), KKH variant SaCas9 (See Kleinstiver et al., Nat Biotechnol. 2015 Dec.; 33(12):1293-1298; WO 2016/141224), Cpf1 (also known as Cas12a, such as AsCpf1, LPCpf1), Cas12f (such as Un1Cas12f1, AsCas12f1) etc. In some embodiments, the RNA-guided nuclease used in the present methods and compositions is a S. aureus Cas9 or a S. pyogenes Cas9, or variants thereof. In some embodiments of this disclosure a Cas9 sequence is modified to include two nuclear localization sequences (NLSs) (e.g., PKKKRKV (SEQ ID NO: 173) at the C-terminus and/or N-terminus of the Cas9 protein, and a mini-polyadenylation signal (or Poly-A sequence). An exemplary NLS is SV40 large T antigen NLS (PKKKRRV (SEQ ID NO: 174)) and nucleoplasmin NLS (KRPAATKKAGQAKKKK (SEQ ID NO: 175)). Other NLSs are known in the art; see, e.g., Cokol et al., EMBO Rep. 2000 Nov. 15; 1(5):411-415; Freitas and Cunha, Curr Genomics. 2009 Dec.; 10(8):550-557. An exemplary polyadenylation signal is TAGCAATAAAGGATCGTTTATTTTCATTGGAAGCGTGTGTTGGTTTTTTGA TCAGGCGCG (SEQ ID NO: 139)). Exemplary S. aureus Cas9 sequences (both nucleotide and peptide) are described in Table 4 of WO 2018/026976, e.g., SEQ ID NOs 10 and 11 therein.

Cas9 nuclease sequences and structures are well known to those of skill in the art (see e.g., “Complete genome sequence of an Ml strain of Streptococcus pyogenes.” Ferretti, J. J. et al., Natl. Acad. Sci. U.S.A. 98:4658-4663(2001); “CRISPR RNA maturation by trans-encoded small RNA and host factor RNase III.” Deltcheva E. et al., Nature 471:602-607(2011); and “Programmable dual-RNA-guided DNA endonuclease in adaptive bacterial immunity.” Jinek M. et al, Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences would be apparent to those of skill in the art based on this disclosure, and such Cas9nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase.

In some embodiments, the compositions and methods disclosed herein use Staphylococcus aureus Cas9 (SaCas9) and corresponding gRNAs. SaCas9 is one of several smaller Cas9 orthologues that are suited for viral delivery (Horvath et al., J Bacteriol 190, 1401-1412 (2008); Ran et al., Nature 520, 186-191 (2015); Zhang et al., Mol Cell 50, 488-503 (2013)). The wild type recognizes a longer NNGRRT PAM that is expected to occur once in every 32 bps of random DNA; or the alternative NNGRRA PAM. Table 1 provides exemplary sequences for the target site in the miR96 locus. Note that the “target site” sequences provided herein are the sequences of the gRNA (although gRNA would have U in place of T).

### Guide RNAs

The genome editing compositions disclosed berein include a guide polynucleotide. In some embodiments, the guide polynucleotide is a guide RNA. An RNA/Cas complex can assist in “guiding” a Cas protein to a target DNA. Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA,” or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference. Cas9 recognizes a short motif in the CRISPR repeat sequences (the PAM or protospacer adjacent motif) to help distinguish self versus non-self.

TABLE 1
Exemplary target site sequences in the miR96 locus

| SEQ ID NO | sgRNA name | targetSeq | Genome editor |
|---|---|---|---|
| 1 | sgm96-40 | CAAGCAAAAATGTGCTAGTCC | KKH-saCas9 |
| 2 | sgm96-41 | CAAGCAAAAATGTGCTAGTAC | KKH-saCas9 |
| 3 | sgm96-42 | CAAGCAAAAATGTGCTAGTGG | KKH-saCas9 |
| 4 | sgm96-43 | CAAGCAAAAATGTGCTAGTGA | KKH-saCas9 |
| 5 | sgm96-44 | CAAGCAAAAATGTGCTAGCGC | KKH-saCas9 |
| 6 | sgm96-45 | CAAGCAAAAATGTGCTAGGGC | KKH-saCas9 |
| 7 | sgm96-46 | GCTAGTACCAAAATOGGCCAA | KKH-saCas9 |
| 8 | sgm96-47 | GCTAGTCCCAAAATCGGCCAA | KKH-saCas9 |
| 9 | sgm96-48 | GCTAGTGACAAAATCGGCCAA | KKH-saCas9 |
| 10 | sgm96-49 | GCTAGTGGCAAAATCGGCCAA | KKH-saCas9 |
| 11 | sgm96-50 | GCTAGGGCCAAAATCGGCCAA | KKH-saCas9 |
| 12 | sgm96-51 | GCTAGCGCCAAAATOGGCCAA | KKH-saCas9 |
| 13 | sgm96-52 | CTGTGAGCAATCACGTGTAGT | KKH-saCas9 |
| 14 | sgm96-53 | AAATGTGCTAGTACCAAAAT | spCas9/Cas9-NG |
| 15 | sgm96-54 | AAATGTGCTAGTCCCAAAAT | spCas9/Cas9-NG |
| 16 | sgm96-55 | AAATGTGCTAGTGACAAAAT | spCas9/Cas9-NG |
| 17 | sgm96-56 | AAATGTGCTAGTGGCAAAAT | spCas9/Cas9-NG |
| 18 | sgm96-57 | AAATGTGCTAGGGCCAAAAT | spCas9/Cas9-NG |
| 19 | sgm96-58 | AAATGTGCTAGCGCCAAAAT | spCas9/Cas9-NG |
| 20 | sgm96-59 | CAAGCAAAAATGTGCTAGTGC | KKH-saCas9 |
| 21 | sgm96-60 | GAAGCAAAAATGTGCTAGTGC | KKH-saCas9 |
| 22 | sgm96-61 | TAAGCAAAAATGTGCTAGTGC | KKH-saCas9 |
| 23 | sgm96-62 | AAAGCAAAAATGTGCTAGTGC | KKH-saCas9 |
| 24 | sgm96-63 | CTAGCAAAAATGTGCTAGTGC | KKH-saCas9 |
| 25 | sgm96-64 | CCAGCAAAAATGTGCTAGTGC | KKH-saCas9 |
| 26 | sgm96-65 | CGAGCAAAAATGTGCTAGTGC | KKH-saCas9 |
| 27 | sgm96-66 | CATGCAAAAATGTGCTAGTGC | KKH-saCas9 |
| 28 | sgm96-67 | CACGCAAAAATGTGCTAGTGC | KKH-saCas9 |
| 29 | sgm96-68 | CAGGCAAAAATGTGCTAGTGC | KKH-saCas9 |
| 30 | sgm96-69 | CAAACAAAAATGTGCTAGTGC | KKH-saCas9 |
| 31 | sgm96-70 | CAATCAAAAATGTGCTAGTGC | KKH-saCas9 |
| 32 | sgm96-71 | CAACCAAAAATGTGCTAGTGC | KKH-saCas9 |
| 33 | sgm96-72 | CAAGAAAAAATGTGCTAGTGC | KKH-saCas9 |
| 34 | sgm96-73 | CAAGTAAAAATGTGCTAGTGC | KKH-saCas9 |
| 35 | sgm96-74 | CAAGGAAAAATGTGCTAGTGC | KKH-saCas9 |
| 36 | sgm96-75 | CAAGCTAAAATGTGCTAGTGC | KKH-saCas9 |
| 37 | sgm96-76 | CAAGCCAAAATGTGCTAGTGC | KKH-saCas9 |
| 38 | sgm96-77 | CAAGCGAAAATGTGCTAGTGC | KKH-saCas9 |
| 39 | sgm96-78 | CAAGCATAAATGTGCTAGTGC | KKH-saCas9 |
| 40 | sgm96-79 | CAAGCACAAATGTGCTAGTGC | KKH-saCas9 |
| 41 | sgm96-80 | CAAGCAGAAATGTGCTAGTGC | KKH-saCas9 |
| 42 | sgm96-81 | CAAGCAATAATGTGCTAGTGC | KKH-saCas9 |
| 43 | sgm96-82 | CAAGCAACAATGTGCTAGTGC | KKH-saCas9 |
| 44 | sgm96-83 | CAAGCAAGAATGTGCTAGTGC | KKH-saCas9 |
| 45 | sgm96-84 | CAAGCAAATATGTGCTAGTGC | KKH-saCas9 |
| 46 | sgm96-85 | CAAGCAAACATGTGCTAGTGC | KKH-saCas9 |
| 47 | sgm96-86 | CAAGCAAAGATGTGCTAGTGC | KKH-saCas9 |
| 48 | sgm96-87 | CAAGCAAAATTGTGCTAGTGC | KKH-saCas9 |
| 49 | sgm96-88 | CAAGCAAAACTGTGCTAGTGC | KKH-saCas9 |
| 50 | sgm96-89 | CAAGCAAAAGTGTGCTAGTGC | KKH-saCas9 |
| 51 | sgm96-90 | CAAGCAAAAAAGTGCTAGTGC | KKH-saCas9 |
| 52 | sgm96-91 | CAAGCAAAAACGTGCTAGTGC | KKH-saCas9 |
| 53 | sgm96-92 | CAAGCAAAAAGGTGCTAGTGC | KKH-saCas9 |
| 54 | sgm96-93 | CAAGCAAAAATATGCTAGTGC | KKH-saCas9 |
| 55 | sgm96-94 | CAAGCAAAAATTTGCTAGTGC | KKH-saCas9 |
| 56 | sgm96-95 | CAAGCAAAAATCTGCTAGTGC | KKH-saCas9 |
| 57 | sgm96-96 | CAAGCAAAAATGAGCTAGTGC | KKH-saCas9 |
| 58 | sgm96-97 | CAAGCAAAAATGCGCTAGTGC | KKH-saCas9 |
| 59 | sgm96-98 | CAAGCAAAAATGGGCTAGTGC | KKH-saCas9 |
| 60 | sgm96-99 | CAAGCAAAAATGTACTAGTGC | KKH-saCas9 |
| 61 | sgm96-100 | CAAGCAAAAATGTTCTAGTGC | KKH-saCas9 |
| 62 | sgm96-101 | CAAGCAAAAATGTCCTAGTGC | KKH-saCas9 |
| 63 | sgm96-102 | CAAGCAAAAATGTGATAGTGC | KKH-saCas9 |
| 64 | sgm96-103 | CAAGCAAAAATGTGTTAGTGC | KKH-saCas9 |
| 65 | sgm96-104 | CAAGCAAAAATGTGGTAGTGC | KKH-saCas9 |
| 66 | sgm96-105 | CAAGCAAAAATGTGCAAGTGC | KKH-saCas9 |
| 67 | sgm96-106 | CAAGCAAAAATGTGCCAGTGC | KKH-saCas9 |
| 68 | sgm96-107 | CAAGCAAAAATGTGCGAGTGC | KKH-saCas9 |
| 69 | sgm96-108 | CAAGCAAAAATGTGCTTGTGC | KKH-saCas9 |
| 70 | sgm96-109 | CAAGCAAAAATGTGCTCGTGC | KKH-saCas9 |
| 71 | sgm96-110 | CAAGCAAAAATGTGCTGGTGC | KKH-saCas9 |
| 72 | sgm96-111 | CAAGCAAAAATGTGCTAATGC | KKH-saCas9 |
| 73 | sgm96-112 | CAAGCAAAAATGTGCTATTGC | KKH-saCas9 |
| 74 | sgm96-113 | CAAGCAAAAATGTGCTACTGC | KKH-saCas9 |
| 75 | sgm96-114 | AAATGTGCTAGTGCCAAAAT | spCas9/Cas9-NG |
| 76 | sgm96-115 | TAATGTGCTAGTGCCAAAAT | spCas9/Cas9-NG |
| 77 | sgm96-116 | CAATGTGCTAGTGCCAAAAT | spCas9/Cas9-NG |
| 78 | sgm96-117 | GAATGTGCTAGTGCCAAAAT | spCas9/Cas9-NG |
| 79 | sgm96-118 | ATATGTGCTAGTGCCAAAAT | spCas9/Cas9-NG |
| 80 | sgm96-119 | ACATGTGCTAGTGCCAAAAT | spCas9/Cas9-NG |
| 81 | sgm96-120 | AGATGTGCTAGTGCCAAAAT | spCas9/Cas9-NG |
| 82 | sgm96-121 | AATTGTGCTAGTGCCAAAAT | spCas9/Cas9-NG |
| 83 | sgm96-122 | AACTGTGCTAGTGCCAAAAT | spCas9/Cas9-NG |
| 84 | sgm96-123 | AAGTGTGCTAGTGCCAAAAT | spCas9/Cas9-NG |
| 85 | sgm96-124 | AAAAGTGCTAGTGCCAAAAT | spCas9/Cas9-NG |
| 86 | sgm96-125 | AAACGTGCTAGTGCCAAAAT | spCas9/Cas9-NG |
| 87 | sgm96-126 | AAAGGTGCTAGTGCCAAAAT | spCas9/Cas9-NG |
| 88 | sgm96-127 | AAATATGCTAGTGCCAAAAT | spCas9/Cas9-NG |
| 89 | sgm96-128 | AAATTTGCTAGTGCCAAAAT | spCas9/Cas9-NG |
| 90 | sgm96-129 | AAATCTGCTAGTGCCAAAAT | spCas9/Cas9-NG |
| 91 | sgm96-130 | AAATGAGCTAGTGCCAAAAT | spCas9/Cas9-NG |
| 92 | sgm96-131 | AAATGCGCTAGTGCCAAAAT | spCas9/Cas9-NG |
| 93 | sgm96-132 | AAATGGGCTAGTGCCAAAAT | spCas9/Cas9-NG |
| 94 | sgm96-133 | AAATGTACTAGTGCCAAAAT | spCas9/Cas9-NG |
| 95 | sgm96-134 | AAATGTTCTAGTGCCAAAAT | spCas9/Cas9-NG |
| 96 | sgm96-135 | AAATGTCCTAGTGCCAAAAT | spCas9/Cas9-NG |
| 97 | sgm96-136 | AAATGTGATAGTGCCAAAAT | spCas9/Cas9-NG |
| 98 | sgm96-137 | AAATGTGTTAGTGCCAAAAT | spCas9/Cas9-NG |
| 99 | sgm96-138 | AAATGTGGTAGTGCCAAAAT | spCas9/Cas9-NG |
| 100 | sgm96-139 | AAATGTGCAAGTGCCAAAAT | spCas9/Cas9-NG |
| 101 | sgm96-140 | AAATGTGCCAGTGCCAAAAT | spCas9/Cas9-NG |
| 102 | sgm96-141 | AAATGTGCGAGTGCCAAAAT | spCas9/Cas9-NG |
| 103 | sgm96-142 | AAATGTGCTTGTGCCAAAAT | spCas9/Cas9-NG |
| 104 | sgm96-143 | AAATGTGCTCGTGCCAAAAT | spCas9/Cas9-NG |
| 105 | sgm96-144 | AAATGTGCTGGTGCCAAAAT | spCas9/Cas9-NG |
| 106 | sgm96-145 | AAATGTGCTAATGCCAAAAT | spCas9/Cas9-NG |
| 107 | sgm96-146 | AAATGTGCTATTGCCAAAAT | spCas9/Cas9-NG |
| 108 | sgm96-147 | AAATGTGCTACTGCCAAAAT | spCas9/Cas9-NG |
| 109 | sgm96-148 | AAATGTGCTAGTGCAAAAAT | spCas9/Cas9-NG |
| 110 | sgm96-149 | AAATGTGCTAGTGCTAAAAT | spCas9/Cas9-NG |
| 111 | sgm96-150 | AAATGTGCTAGTGCGAAAAT | spCas9/Cas9-NG |
| 112 | sgm96-151 | AAATGTGCTAGTGCCTAAAT | spCas9/Cas9-NG |
| 113 | sgm96-152 | AAATGTGCTAGTGCCGAAAT | spCas9/Cas9-NG |
| 114 | sgm96-153 | AAATGTGCTAGTGCCCAAAT | spCas9/Cas9-NG |
| 115 | sgm96-154 | AAATGTGCTAGTGCCATAAT | spCas9/Cas9-NG |
| 116 | sgm96-155 | AAATGTGCTAGTGCCACAAT | spCas9/Cas9-NG |
| 117 | sgm96-156 | AAATGTGCTAGTGCCAGAAT | spCas9/Cas9-NG |
| 118 | sgm96-157 | AAATGTGCTAGTGCCAATAT | spCas9/Cas9-NG |
| 119 | sgm96-158 | AAATGTGCTAGTGCCAACAT | spCas9/Cas9-NG |
| 120 | sgm96-159 | AAATGTGCTAGTGCCAAGAT | spCas9/Cas9-NG |
| 121 | sgm96-160 | AAATGTGCTAGTGCCAAATT | spCas9/Cas9-NG |
| 122 | sgm96-161 | AAATGTGCTAGTGCCAAACT | spCas9/Cas9-NG |
| 123 | sgm96-162 | AAATGTGCTAGTGCCAAAGT | spCas9/Cas9-NG |
| 124 | sgm96-163 | AAATGTGCTAGTGCCAAAAA | spCas9/Cas9-NG |
| 125 | sgm96-164 | AAATGTGCTAGTGCCAAAAC | spCas9/Cas9-NG |
| 126 | sgm96-165 | AAATGTGCTAGTGCCAAAAG | spCas9/Cas9-NG |
| 127 | sgm96-166 | AAATGTGCTAGTTCCAAAAT | spCas9/Cas9-NG |
| 128 | sgm96-167 | AAATGTGCTAGAGCCAAAAT | spCas9/Cas9-NG |
| 129 | sgm96-12 | AAATGTGCTAGTTCCAAAAT | spCas9/Cas9-NG |
| 130 | sgm96-1 | CAAGCAAAAATGTGCTAGTTC | KKH-saCas9 |
| 131 | sgm96-2 | CAAGCAAAAATGTGCTAGTGT | KKH-saCas9 |
| 132 | sgm96-3 | CAAGCAAAAATGTGCTAGAGC | KKH-saCas9 |
| 133 | sgm96-4 | TCTCCGCTCTGAGCAATCACG | KKH-saCas9 |
| 134 | sgm96-5 | GCTAGTTCCAAAATCGGCCAA | KKH-saCas9 |
| 135 | sgm96-6 | GCTAGTGTCAAAATCGGCCAA | KKH-saCas9 |
| 136 | sgm96-7 | GCTAGAGCCAAAATOGGCCAA | KKH-saCas9 |
| 137 | sgm96-8 | CTCTGAGCAATCACGTGCAGT | KKH-saCas9 |
| 138 | sgm96-9 | AGTTCCAAAATCGGCCAAGC | Cas9-NG |
| 139 | sgm96-10 | GCTAGTTCCAAAATCGGCCA | Cas9-NG |
| 140 | sgm96-11 | AATGTGCTAGTTCCAAAATC | Cas9-NG |
| 141 | sgm96-13 | GCTTGGCCGATTTTGGAACT | Cas9-NG |
| 142 | sgm96-14 | AGTGTCAAAATCGGCCAAGC | Cas9-NG |
| 143 | sgm96-15 | GCTAGTGTCAAAATOGGCCA | Cas9-NG |
| 144 | sgm96-16 | AATGTGCTAGTGTCAAAATC | Cas9-NG |
| 145 | sgm96-17 | AAATGTGCTAGTGTCAAAAT | spCas9/Cas9-NG |
| 146 | sgm96-18 | GCTTGGCCGATTTTGACACT | Cas9-NG |
| 147 | sgm96-19 | AGAGCCAAAATCGGCCAAGC | Cas9-NG |
| 148 | sgm96-20 | GCTAGAGCCAAAATCGGCCA | Cas9-NG |
| 149 | sgm96-21 | AATGTGCTAGAGCCAAAATC | Cas9-NG |
| 150 | sgm96-22 | AAATGTGCTAGAGCCAAAAT | spCas9/Cas9-NG |
| 151 | sgm96-23 | GCTTGGCCGATTTTGGCTCT | Cas9-NG |
| 152 | sgm96-24 | CTGCACGTGATTGCTCAGAG | spCas9/Cas9-NG |
| 153 | sgm96-25 | CACTGCACGTGATTGCTCAG | Cas9-NG |
| 154 | sgm96-26 | GGCACTGCACGTGATTGCTC | Cas9-NG |
| 155 | sgm96-27 | ATATTGGCACTGCACGTGAT | Cas9-NG |
| 156 | sgm96-28 | TCCCATATTGGCACTGCACG | Cas9-NG |
| 157 | sgm96-29 | CTCCGCTCTGAGCAATCACG | Cas9-NG |
| 158 | sgm96-30 | CGCTCTGAGCAATCACGTGC | Cas9-NG |
| 159 | sgm96-31 | CTCTGAGCAATCACGTGCAG | Cas9-NG |
| 160 | sgm96-32 | ATATTGGCACTGCACGTGAT | Cas9-NG |
| 161 | sgm96-33 | ATCACGTGCAGTGCCAATAT | spCas9/Cas9-NG |
| 162 | sgm96-34 | TCACGTGCAGTGCCAATATG | Cas9-NG |
| 163 | sgm96-35 | AATCACGTGCAGTGCCAATA | CBE-spCas9/CBE-Cas9-NG |
| 164 | sgm96-36 | ATCACGTGCAGTGCCAATAT | CBE-spCas9/CBE-Cas9-NG |
| 165 | sgm96-37 | TCACGTGCAGTGCCAATATG | CBE-Cas9-NG |
| 166 | sgm96-38 | TTTTGACACTAGCACATTTT | ABE-Cas9-NG |
| 167 | sgm96-39 | GCCATCTGCTTGGCCGATTT | Prime editing |

In some embodiments, the guide polynucleotide is at least one single guide RNA (“sgRNA” or “gNRA”). In some embodiments, the guide polynucleotide is at least one tracrRNA, In some embodiments, the guide polynucleotide does not require protospacer adjacent motif (PAM) sequence to guide the polynucleotide-programmable DNA-binding domain (e.g., Cas9 or Cpf1) to the target nucleotide sequence. The polynucleotide programmable nucleotide binding domain (e.g., a CRISPR-derived domain) of the genome editing systems disclosed herein can recognize a target polynucleotide sequence by associating with a guide polynucleotide. A guide polynucleotide (e.g., gRNA) is typically single-stranded and can be programmed to site-specifically bind (i.e., via complementary base pairing) to a target sequence of a polynucleotide, thereby directing a base editor that is in conjunction with the guide nucleic acid to the target sequence. A guide polynucleotide can be DNA. A guide polynucleotide can be RNA. In some embodiments, the guide polynucleotide comprises natural nucleotides (e.g., adenosine). In some embodiments, the guide polynucleotide comprises non-natural (or unnatural) nucleotides (e.g., peptide nucleic acid or nucleotide analogs). In some embodiments, the targeting region of a guide nucleic acid sequence can be at least 15, 16, 17, 18, 19, 20, 21, 22, 23, 24, 25, 26, 27, 28, 29, or 30 nucleotides in length. A targeting region of a guide nucleic acid can be between 10-30 nucleotides in length, or between 15-25 nucleotides in length, or between 15-20 nucleotides in length.

The guide RNAs, e.g., sgRNAs, used in the disclosed methods and compositions comprises a guide sequence targeting the miR96 locus. Exemplary guide sequences targeting the miR96 locus are shown in Table 1 at SEQ ID NOs: 1-130. Guide sequences useful in the guide RNA compositions and methods described herein are shown in Table 1 and throughout the application. Note that in the sequences provided here, the actual sgRNA would have U in place of T.

In the case of a sgRNA, the guide sequences of Table 1 may further comprise additional nucleotides to form a sgRNA, e.g., with the following exemplary nucleotide sequence following the 3′ end of the guide sequence, wherein the sgRNA has a custom-designed short crRNA component selected from Table 1 followed by the tracrRNA component, for example: GUUUUAGUACUCUGGAAACAGAAUCUACUAAAACAAGGCAAAAUGCCGUGUUUAUCUCGUCA ACUUGUUGGCGAGAUUUU (SEQ ID NO: 168) in the 5′ to 3′ orientation. Any one of the guide sequences of Table 1 may be used in combination with any one of the sequences of Table 2 to form an sgRNA for use in the methods and compositions disclosed herein.

In some embodiments, the sgRNA for use with the genome editing system disclosed herein comprises a polynucleotide sequence that is 80%, 85%, 90%, 95%, 99%, or 100% identical to SEQ ID NO: 129. In some embodiments, the sgRNA for use with the genome editing system disclosed herein comprises a polynucleotide sequence that is 80%, 85%, 90%, 95%, 99%, or 100% identical to SEQ ID NO: 127. In some embodiments, the sgRNA for use with the genome editing system disclosed berein comprises a polynucleotide sequence that is 80%, 85%, 90%, 95%, 99%, or 100% identical to SEQ ID NO: 128.

In some embodiments, the sgRNA for use with the genome editing system disclosed herein comprises a polynucleotide sequence that is 80%, 85%, 90%, 95%, 99%, or 100% identical to SEQ ID NO: 168. In some embodiments, the sgRNA for use with the genome editing system disclosed herein comprises a polynucleotide sequence that is 80%, 85%, 90%, 95%, 99%, or 100% identical to SEQ ID NO: 169. In some embodiments, the sgRNA for use with the genome editing system disclosed berein comprises a polynucleotide sequence that is 80%, 85%, 90%, 95%, 99%, or 100% identical to SEQ ID NO: 170. In some embodiments, the sgRNA for use with the genome editing system disclosed herein comprises a polynucleotide sequence that is 80%, 85%, 90%, 95%, 99%, or 100% identical to SEQ ID NO: 171.

In some embodiments, the sgRNA for use with the genome editing system disclosed herein comprises a polynucleotide sequence that is 80%, 85%, 90%, 95%, 99%, or 100% identical to SEQ ID NO: 129 and further comprises a polynucleotide sequence that is 80%, 85%, 90%, 95%, 99%, or 100% identical to SEQ ID NO: 171, In some embodiments, the genome editing system comprises an spCas9 nuclease and an sgRNA comprising the polynucleotide sequences set forth in SEQ ID NOs: 129 and 171. In some embodiments, the sgRNA for use with the genome editing system disclosed herein comprises a polynucleotide sequence that is 80%, 85%, 90%, 95%, 99%, or 100% identical to SEQ ID NO: 127 and further comprises a polynucleotide sequence that is 80%, 85%, 90%, 95%, 99%, or 100% identical to SEQ ID NO: 171. In some embodiments, the genome editing system comprises an spCas9 nuclease and an sgRNA comprising the polynucleotide sequences set forth in SEQ ID NOs: 127 and 171. In some embodiments, the sgRNA for use with the genome editing system disclosed herein comprises a polynucleotide sequence that is 80%, 85%, 90%, 95%, 99%, or 100% identical to SEQ ID NO: 128 and further comprises a polynucleotide sequence that is 80%, 85%, 90%, 95%, 99%, or 100% identical to SEQ ID NO: 171. In some embodiments, the genome editing system comprises an spCas9 nuclease and an sgRNA comprising the polynucleotide sequences set forth in SEQ ID NOs: 128 and 171.

TABLE 2

| SEQ | | |
|---|---|---|
| ID | | |
| NO | Sequence type | |
| 168 | Original KKH- | GUUUUAGUACUCUGGAAACAGAAU |
| | saCas9 sgRNA | CUACUAAAACAAGGCAAAAUGCCG |
| | | UGUUUAUCUCGUCAACUUGUUGGC |
| | | GAGAUUUU |
| 169 | Optimized KKH- | GUUAUAGUACUCUGUAAUGAAAAU |
| | saCas9 sgRNA | UACAGAAUCUACUAUAACAAGGCA |
| | | AAAUGCCGUGUUUAUCUCGUCAAC |
| | | UUGUUGGCGAGAUUUU |
| 170 | Original | GUUUUAGAGCUAGAAAUAGCAA |
| | spCas9 sgRNA | GUUAAAAUAAGGCUAGUCCGUU |
| | | AUCAACUUGAAAAAGUGGCACC |
| | | GAGUCGGUGCUUUUUU |
| 171 | Optimized | GUUUCAGAGCUAUGCUGGAAACAG |
| | spCas9 sgRNA | CAUAGCAAGUUGAAAUAAGGCUAG |
| | | UCCGUUAUCAACUUGAAAAAGUGG |
| | | CACCGAGUCGGUGCUUUUUU |

### AAV Delivery Systems

The methods include delivery of a CRISPR/Cas9 genome editing system, including a nucleic acid-guided nuclease and one or more guide RNAs, to a subject in need thereof. The delivery methods can include, e.g., viral delivery, preferably using an adeno-associated virus (AAV) vector that encodes the nucleic acid-guided nuclease and one or more guide RNA(s). AAV is a naturally occurring defective virus that requires another virus, such as an adenovirus or a herpes virus, as a helper virus for efficient replication and a productive life cycle. (For a review, see Muzyczka et al., Curr. Topics in Micro and Immunol. 158:97-129 (1992)). AAV vectors efficiently transduce various cell types and can produce long-term expression of transgenes in vivo. AAV vectors have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. For example, AAV2, AAV5, AAV2/5, AAV2/8 and AAV2/7 vectors have been used to introduce DNA into photoreceptor cells (see, e.g., Pang et al., Vision Research 2008, 48(3):377-385; Khani et al., Invest Ophthalmol Vis Sci. 2007 Sep.;48(9):3954-61; Allocca et al., J. Virol. 2007 81(20):11372-11380). In some embodiments, the AAV vector can include (or include a sequence encoding) an AAV capsid polypeptide described in PCT/US2014/060163; for example, a virus particle comprising an AAV capsid polypeptide having an amino acid sequence selected from the group consisting of SEQ ID NOs: 1, 3, 5, 7, 9, 11, 13, 15, and 17 of PCT/US2014/060163, and a nucleic acid-guided nuclease sequence and guide RNA sequence as described herein. In some embodiments, the AAV capsid polypeptide is an Anc80 polypeptide, e.g., Anc80L27; Anc80L59; Anc80L60; Anc80L62; Anc80L65; Anc80L33; Anc80L36; or Anc80L44. In some embodiments, the AAV incorporates inverted terminal repeats (ITRs) derived from the AAV2 serotype. Exemplary left and right ITRs are presented in Table 6 of WO 2018/026976. It should be noted, however, that numerous modified versions of the AAV2 ITRs are used in the field, and the ITR sequences shown below are exemplary and are not intended to be limiting. Modifications of these sequences are known in the art, or will be evident to skilled artisans, and are thus included in the scope of this disclosure.

The approaches described herein include the use of retroviral vectors, adenovirus-derived vectors, and/or adeno-associated viral vectors as recombinant gene delivery systems for the transfer of exogenous genes in vivo, particularly into humans. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals.

In certain embodiments, an adenovirus can be used in accordance with the methods described herein. The genome of an adenovirus can be manipulated such that it encodes and expresses a gene product of interest but is inactivated in terms of its ability to replicate in a normal lytic viral life cycle. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors.

Adeno-associated virus is a naturally occurring defective virus that requires another virus, such as an adenovirus or a herpes virus, as a helper virus for efficient replication and a productive life cycle. It is also one of the few viruses that may integrate its DNA into non-dividing cells, and exhibits a high frequency of stable integration.

Expression of the nucleic acid-guided nuclease, e.g., Cas9, spCas9, scCas9++, LZ3 Cas9, KKH-saCas9 or sauriCas9, can be driven by a promoter known in the art. In some embodiments, expression of the nuclease is driven by a cytomegalovirus (CMV) promoter. Modifications of the promoter sequence may be possible or desirable in certain applications, and such modifications are within the scope of this disclosure.

Expression of the gRNAs in the AAV vector is driven by a promoter known in the art. In some embodiments, a polymerase III promoter, such as a human U6 promoter. An exemplary U6 promoter sequence is presented below:

(SEQ ID NO: 140)
AAGGTCGGGCAGGAAGAGGGCCTATTTCCCATGATTCCTTCATAT
TTGCATATACGATACAAGGCTGTTAGAGAGATAATTAGAATTAAT
TTGACTGTAAACACAAAGATATTAGTACAAAATACGTGACGTAGA
AAGTAATAATTTCTTGGGTAGTTTGCAGTTTTAAAATTATGTTTT
AAAATGGACTATCATATGCTTACCGTAACTTGAAAGTATTTCGAT
TTCTTGGCTTTATATATCTTGTGGAAAGGACGAAACACC.

In some embodiments, the nucleic acid or AAV vector shares at least 80%, 85%, 90%, 95%, 96%, 97%, 98%, 99% or greater sequence identity with one of the nucleic acids or AAV vectors recited above.

The AAV genomes described above can be packaged into AAV capsids (for example, AAV5 capsids), which capsids can be included in compositions (such as pharmaceutical compositions) and/or administered to subjects. An exemplary pharmaceutical composition comprising an AAV capsid according to this disclosure can include a pharmaceutically acceptable carrier such as balanced saline solution (BSS) and one or more surfactants (e.g., Tween 20) and/or a thermosensitive or reverse-thermosensitive polymer (e.g., pluronic). Other pharmaceutical formulation elements known in the art may also be suitable for use in the compositions described here.

Compositions comprising AAV vectors according to this disclosure can be administered to subjects by any suitable means, including without limitation injection, for example, injection through the round window. The concentration of AAV vector within the composition is selected to ensure, among other things, that a sufficient AAV dose is administered to the inner ear of the subject, taking account of dead volume within the injection apparatus and the relatively limited volume that can be safely administered. Suitable doses may include, for example, 1×10¹¹ viral genomes (vg)/mL, 2×10¹¹ viral genomes (vg)/mL, 3×10¹¹ viral genomes (vg)/mL, 4×10¹¹ viral genomes (vg)/mL, 5×10¹¹ viral genomes (vg)/mL, 6×10¹¹ viral genomes (vg)/mL, 7×10¹¹ viral genomes (vg)/mL, 8×10¹¹ viral genomes (vg)/mL, 9×10¹¹ viral genomes (vg)/mL, 1×10¹² vg/mL, 2×10¹² viral genomes (vg)/mL, 3×10¹² viral genomes (vg)/mL, 4×10¹² viral genomes (vg)/mL, 5×10¹² viral genomes (vg)/mL, 6×10¹² viral genomes (vg)/mL, 7×10¹² viral genomes (vg)/mL, 8×10¹² viral genomes (vg)/mL, 9×10¹² viral genomes (vg)/mL, 1×10¹³ vg/mL, 2×10¹³ viral genomes (vg)/mL, 3×10¹³ viral genomes (vg)/mL, 4×10¹³ viral genomes (vg)/mL, 5×10¹³ viral genomes (vg)/mL, 6×10¹³ viral genomes (vg)/mL, 7×10¹³ viral genomes (vg)/mL, 8×10¹³ viral genomes (vg)/mL, or 9×10¹³ viral genomes (vg)/mL. Any suitable volume of the composition may be delivered to the cochlear space. In some instances, the volume is selected to form a bleb in the cochlear space, for example 1 microliter, 10 microliters, 50 microliters, 100 microliters, 150 microliters, 200 microliters, 250 microliters, 300 microliters, etc.

For delivery to the inner car, injection to the cochlear duct, which is filled with high potassium endolymph fluid, can provide direct access to hair cells. However, alterations to this delicate fluid environment may disrupt the endocochlear potential, heightening the risk for injection-related toxicity. The perilymph-filled spaces surrounding the cochlear duct, scala tympani and scala vestibuli, can be accessed from the middle ear, either through the oval or round window membrane (RWM). The RWM, which is the only non-bony opening into the inner ear, is relatively easily accessible in many animal models and administration of viral vector using this route is well-tolerated. Administration through the oval window or across the tympanic membrane can also be used. See, e.g., WO2017100791 and U.S. Pat. No. 7,206,639.

In certain embodiments, delivery of the compositions disclosed herein may occur by use of liposomes, nanocapsules, microparticles, microspheres, lipid particles, vesicles, optionally mixing with cell penetrating polypeptides, and the like, for the introduction of the compositions of the present invention into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

For pre-clinical development purposes, systems, compositions, nucleotides and vectors according to this disclosure can be evaluated in vitro using a human or mouse engineered cell lines, or in vivo using an animal model such as a mouse, rabbit, pig, nonhuman primate, etc.

Explants, or cells derived from inner organs of animal models, are particularly useful for studying the expression of gRNAs and/or Cas9 following viral transduction, and for studying genome editing over comparatively short intervals. These models also permit higher throughput than may be possible in animal models, and can be predictive of expression and genome editing in animal models and subjects. Small (mouse, rat) and large animal models (such as rabbit, pig, nonhuman primate) can be used for pharmacological and/or toxicological studies and for testing the systems, nucleotides, vectors and compositions of this disclosure under conditions and at volumes that approximate those that will be used in clinic. Because model systems are selected to recapitulate relevant aspects of human anatomy and/or physiology, the data obtained in these systems will generally (though not necessarily) be predictive of the behavior of AAV vectors and compositions according to this disclosure in human and animal subjects.

### Methods of Treatment

The methods described herein include methods for the treatment of disorders associated with mutations in the miR96 locus.

In some embodiments, the disorder autosomal dominant progressive nonsyndromic all-frequency hearing loss, e.g., DFNA50. Subjects with DFNA50 typically have sensorineural progressive hearing loss of all frequencies, with hearing loss as the only clinical feature (i.e., nonsyndromic). Age of onset is typically around 12 years of age, with initial hearing loss being mild and progressing to severe or profound by the seventh decade of life. Generally, the methods of treatment disclosed herein include administering a therapeutically effective amount of a genome editing system as described herein, to a subject who is in need of, or who has been determined to be in need of, such treatment. The term “genome editing system” refers to any system having RNA-guided DNA editing activity. Genome editing systems of the present disclosure include at least two components adapted from naturally occurring CRISPR systems: a gRNA and an RNA-guided nuclease. These two components form a complex that is capable of associating with a specific nucleic acid sequence in a cell and editing the DNA in or around that nucleic acid sequence, for example by making one or more of a single-strand break (an SSB or nick), a double-strand break (a DSB) and/or a base substitution. See, e.g., WO2018/026976 for a description of exemplary genome editing systems.

As used in this context, to “treat” means to ameliorate at least one symptom of the disorder associated with a mutation of the miR96 locus. Often, these mutations result in progressive nonsyndromic hearing loss; thus, a treatment comprising administration of a therapeutic gene editing system as described herein can result in a reduction in hearing impairment; a reduction in the rate of progression of hearing loss; and/or a return or approach to normal hearing. Hearing can be tested using known methods, e.g., audiology testing.

The methods can be used to treat any subject (e.g., a mammalian subject, preferably a human subject) who has a mutation of the miR96 locus. The therapeutic gene editing system as described herein can disrupt the mutant allele associated with the disease. As used herein, an “allele” is one of a pair or series of genetic variants of a polymorphism (also referred to as a mutation) at a specific genomic location. As used herein, “genotype” refers to the diploid combination of alleles for a given genetic polymorphism. A homozygous subject carries two copies of the same allele and a heterozygous subject carries two different alleles. Methods for identifying subjects with such mutations are known in the art; see, e.g., Yan et al., J Hum Genet. 2009 Dec.; 54(12): 732-738; Leroy et al., Exp Eye Res. 2001 May;72(5):503-9; or Consugar et al., Genet Med. 2015 Apr.;17(4):253-261. For example, gel electrophoresis, capillary electrophoresis, size exclusion chromatography, sequencing, and/or arrays can be used to detect the presence or absence of the allele or genotype. Amplification of nucleic acids, where desirable, can be accomplished using methods known in the art, e.g., PCR. In one example, a sample (e.g., a sample comprising genomic DNA), is obtained from a subject. The DNA in the sample is then examined to identify or detect the presence of an allele or genotype as described herein. The allele or genotype can be identified or determined by any method described herein, e.g., by Sanger sequencing or Next Generation Sequencing (NGS). Other methods can include hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular mutation (also referred to as a polymorphic variant).

Other methods of nucleic acid analysis can include direct manual sequencing (Church and Gilbert, Proc. Natl. Acad. Sci. USA 81:1991-1995 (1988); Sanger et al., Proc. Natl. Acad. Sci. USA 74:5463-5467 (1977); Beavis et al., U.S. Pat. No. 5,288,644); automated fluorescent sequencing; single-stranded conformation polymorphism assays (SSCP) (Schafer et al., Nat. Biotechnol. 15:33-39 (1995)); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)); denaturing high performance liquid chromatography (DHPLC, Underhill et al., Genome Res. 7:996-1005 (1997)); infrared matrix-assisted laser desorption/ionization (IR-MALDI) mass spectrometry (WO 99/57318); mobility shift analysis (Orita et al., Proc. Natl. Acad. Sci. USA 86:2766-2770 (1989)); restriction enzyme analysis (Flavell et al., Cell 15:25 (1978); Geever et al., Proc. Natl. Acad. Sci, USA 78:5081 (1981)); quantitative real-time PCR (Raca et al., Genet Test 8(4):387-94 (2004)); heteroduplex analysis; chemical mismatch cleavage (CMC) (Cotton et al., Proc. Natl. Acad. Sci. USA 85:4397-4401 (1985)); RNase protection assays (Myers et al., Science 230:1242 (1985)); use of polypeptides that recognize nucleotide mismatches, e.g., E. coli mutS protein; allele-specific PCR, and combinations of such methods. See, e.g., Gerber et al., U.S. Patent Publication No. 2004/0014095 which is incorporated herein by reference in its entirety.

In certain aspects, the present disclosure provides AAV vectors encoding CRISPR/Cas9 genome editing systems, and provides the use of such vectors to treat miR96-associated disease. Exemplary AAV vector genomes are described in WO2019/183641, which illustrates certain fixed and variable elements of these vectors: inverted terminal repeats (ITRs), one or more gRNA sequences and promoter sequences to drive their expression, a nuclease coding sequence and another promoter to drive its expression (an exemplary construct for use in the methods described herein could include, for example 2 gRNA or only 1 gRNA and U6). Each of these elements is discussed in detail herein. A single vector can be used to deliver a Cas9 and one or more gRNAs, or a plurality of vectors can be used, e.g., wherein one vector is used to deliver Cas9, and another vector or vectors is used to deliver one or more gRNAs (e.g., one vector for one gRNA, one vector for two gRNAs, one vector for three gRNAs, or three vectors for each of three gRNAs). Other arrangements are also possible, including splitting the Cas9 across two AAV.

In some embodiments, the nucleic acid compositions described herein, that include a gRNA and a nucleic acid encoding an RNA-guided nuclease encoded on one or more vectors, are formulated in or administered via a lipid nanoparticle (LNP); sce e.g., WO2017173054A1 and WO2019067992A1, the contents of which are hereby incorporated by reference in their entireties. Any LNP known to those of skill in the art to be capable of delivering nucleotides to subjects may be utilized with the guide RNAs described herein and the nucleic acid encoding an RNA-guided nuclease.

In some embodiments, the guide RNA and the nucleic acid encoding an RNA-guided nuclease are administered in an LNP described herein, such as an LNP that includes a CCD lipid (e.g., an amine lipid, such as lipid A), a helper lipid (e.g., cholesterol), a stealth lipid (e.g., a PEG lipid, such as PEG2k-DMG), and optionally a neutral lipid (e.g., DSPC).

Disclosed herein are various embodiments of LNP formulations for RNAs, including CRISPR/Cas cargoes. Such LNP formulations may include (i) a CCD lipid, such as an amine lipid, (ii) a neutral lipid, (iii) a helper lipid, and (iv) a stealth lipid, such as a PEG lipid. Some embodiments of the LNP formulations include an amine lipid, along with a helper lipid, a neutral lipid, and a stealth lipid such as a PEG lipid. In some embodiments, the LNP formulations include less than 1 percent neutral phospholipid. In some embodiments, the LNP formulations include less than 0.5 percent neutral phospholipid. A “lipid nanoparticle” could be a particle that comprises a plurality of (i.e. more than one) lipid molecules physically associated with each other by intermolecular forces. CCD Lipids, Amine Lipids, Neutral Lipids, and other lipids that can be used in the LNP formulations disclosed herein are described in WO2020198697, WO2015006747, WO2016118724, and WO2021026358, each of which is incorporated herein in its entirety.

Further technologies that can be used for delivery of the compositions of this disclosure include those that utilize encapsulation by biodegradable polymers, liposomes, or nanoparticles. In some embodiments, the compositions of this disclosure are administered in any suitable delivery vehicle, including, but not limited to, polymers, engineered viral particles (e.g., adeno-associated virus), exosomes, liposomes, supercharged proteins, implantable devices, or red blood cells. Suitable delivery methods are described in U.S. Pat. Nos. 10,851,357, 10,709,797, and US20170349914, each of which is incorporated herein in its entirety.

### EXAMPLES

The compositions and methods disclosed herein are further described in the following examples, which do not limit the scope of the claims.

### Methods

The following materials and methods were used in the following examples.

### Animals

All animals were bred and housed in Mass Eye and Ear Infirmary (MEEI). All studies involving animals were approved by the Harvard Medical School Standing Committee on Animals and the Mass Eye and Ear Infirmary Animal Care and Use Committee. All mice were housed in a room maintained on a 12 hour light and dark cycle with ad libitum access to standard rodent diet.

### Plasmid Construction

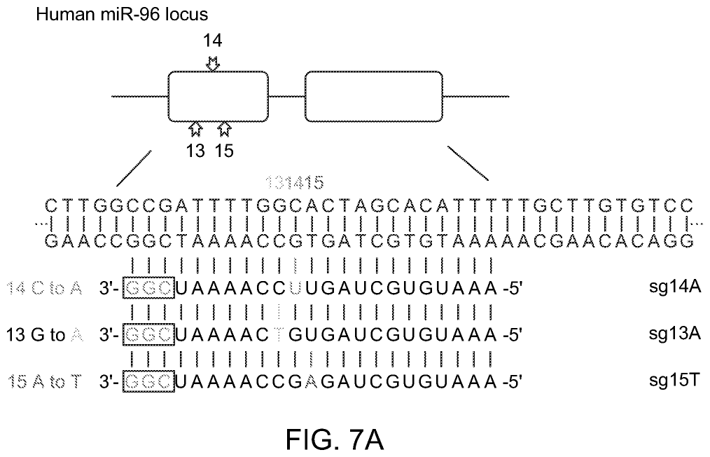

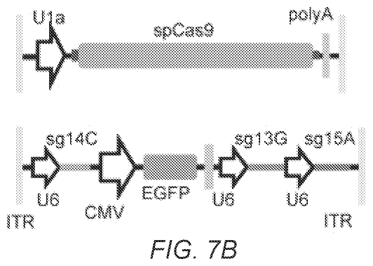

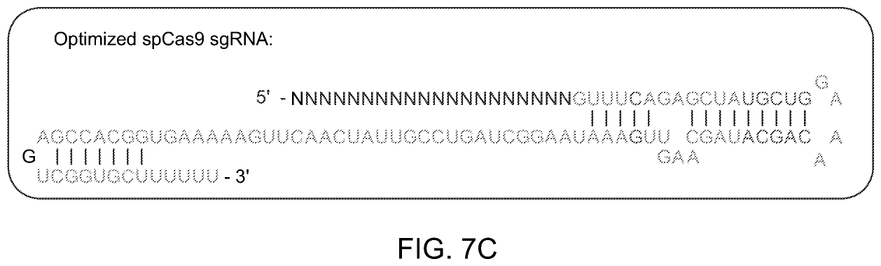

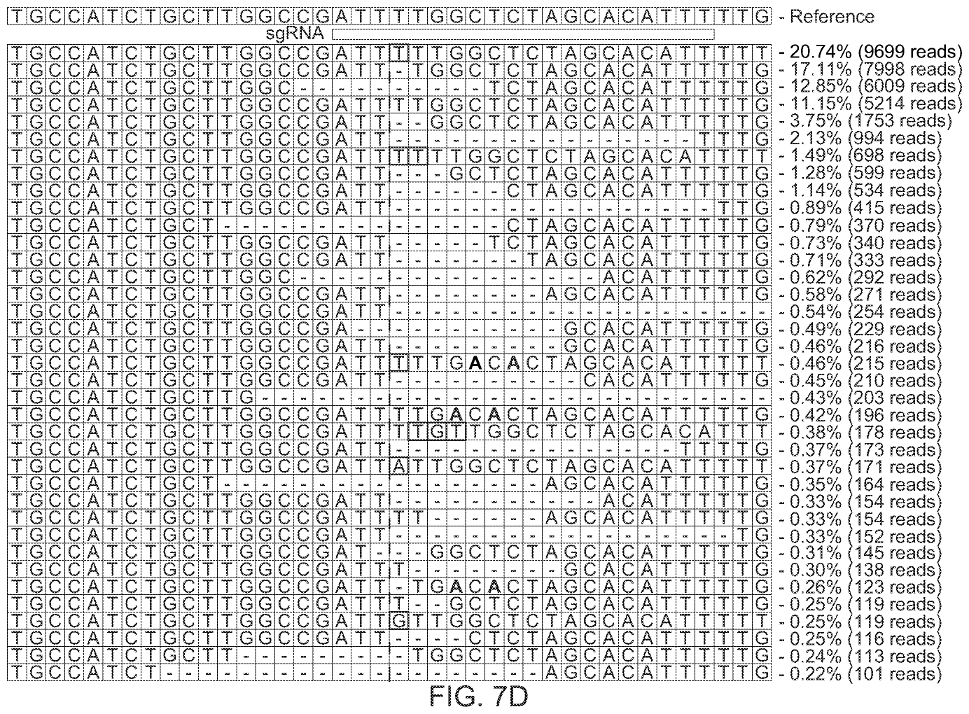

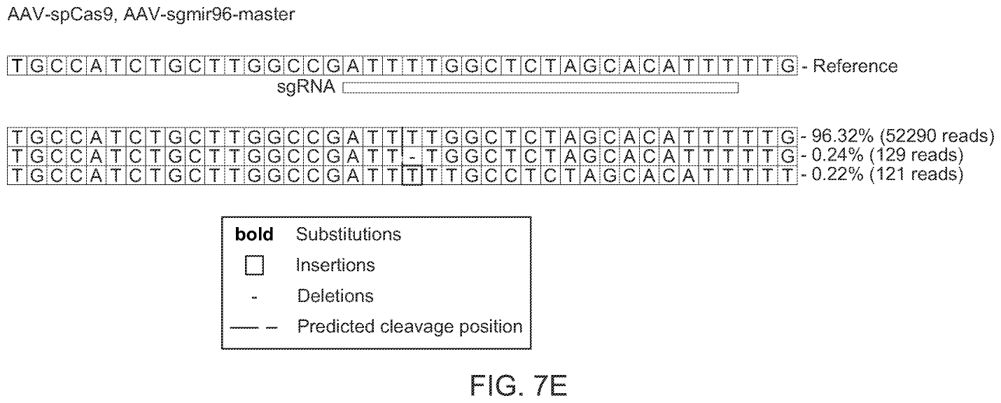

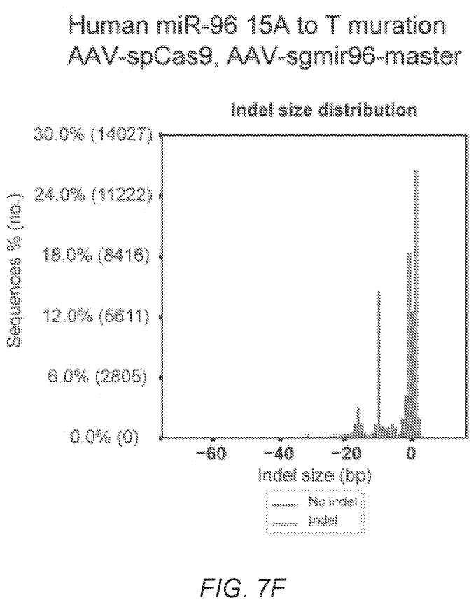

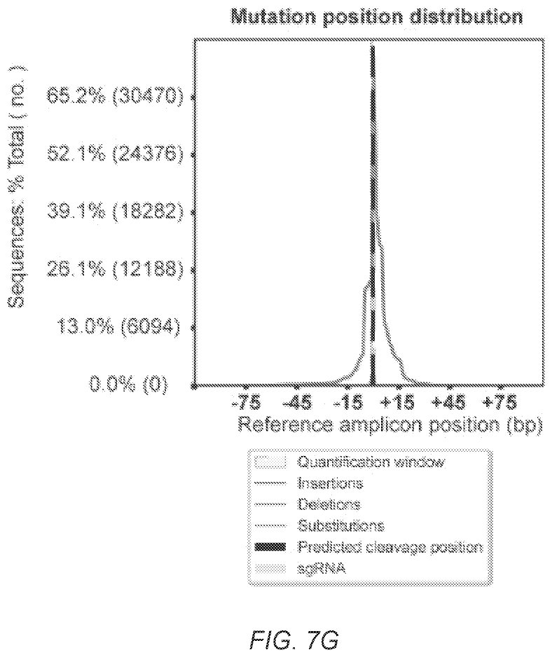

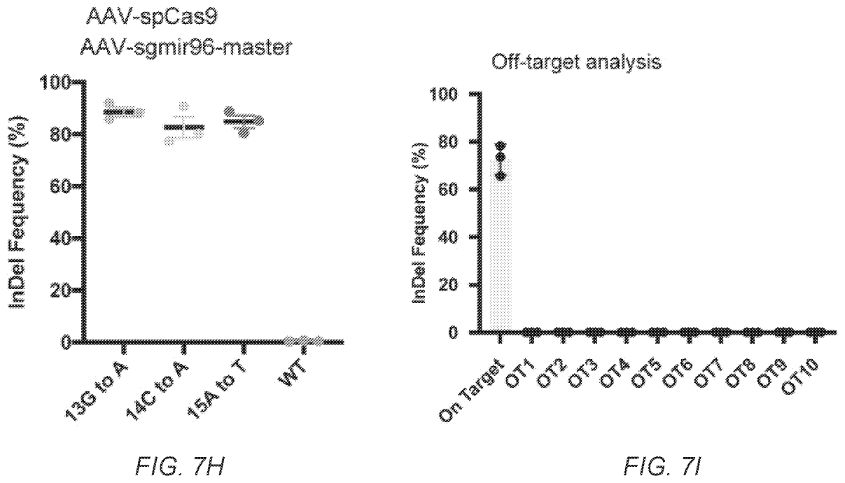

pMax-spCas9 was obtained commercially. U6-sgRNA sequence was obtained from PX458 (Addgene 48138), and other Cas9 nucleases, including scCas9++ (Addgene 155011), KKH-saCas9 (Addgene 70707), sauriCas9 (Addgene 135964), LZ3 Cas9 (Addgene 140561) cDNA were acquired from Addgene. Vectors for in vitro screening were constructed via Gibson assembly (NEB, E2611S) of PCR-amplified fragments. AAV vectors were based on PX601 (Addgene 61591), for AAV-U1a-opti-spCas9-PA, the U1a promoter sequence was obtained from pAAV.pU1a-SpCas9 (Addgene 121507). AAV-sgmir96-Master (FIG. 7B) was based on pAAV-U6-sgRNA-CMV-GFP (Addgene 85451). Plasmids encoding recombinant AAV (rAAV) genomes were cloned by Gibson assembly. All plasmids were purified using Plasmid Plus Midiprep or Maxiprep kits (Qiagen).

### AAV Production

AAV plasmids containing “CMV-KKH-SaCas9-PA” cassette, “U6-sgRNA-4” or “U6-sgCtrl” cassette was sequenced before packaging into AAV2/2. Vector titer was 4.76×10¹² vg/ml for KKH-saCas9/sgRNA-4 and 3.55×10¹² vg/ml for KKH-saCas9/sgCtrl as determined by qPCR specific for the inverted terminal repeat of the virus.

### Isolation and Culture of Primary Fibroblast from Mice

MiR-96^{s1334/+}, Ai14 (CAG-STOP-tdTomato) and wild type primary fibroblasts were obtained from adult mice. Mice were cuthanized and cleaned with 70% ethanol. The dorsal skin (˜1 cm diameter) of the mice was excised after trimming the hair and sterilizing the area with 70 percent alcohol. The collected skin was rinsed with DPBS containing 200 mg/mL streptomycin and 200 U/mL penicillin, Subcutaneous fat was removed by forceps. Subsequently, the samples were cut into small fragments using a sterile scalpel and incubated with Dispase II (Sigma-Aldrich, USA) for overnight at 4° C. Following the incubation, the epidermal and dermal layers of the skin were separated using forceps. The dermal portion is further subjected to incubation with type I collagenase (1 mg/ml Gibco, USA) for 2 hours at 37 degrees. Warm culture medium (1:1 DMEM: F12 medium (ThermoFisher) with 10% fetal bovine serum (FBS) (ThermoFisher) and 100 U/ml penicillin+streptomycin (ThermoFisher) was added to stop the enzyme digestion. The resulting cell suspension was strained using a 40-micron strainer and centrifuged at 950 rpm to obtain the cell pellet. The cell pellet was seeded in T-75 flask containing DMEM high glucose media (Gibco, USA) containing 10% FBS (Gibco, USA), then incubated at 37° C. with 5% CO₂ and 3% O₂. Fibroblasts were cultured for about 2-3 days to reach ˜90% confluence, then passaged in T75 flasks with TrypLE Express and cultured in DMEM:F12 medium (ThermoFisher) with 10% fetal bovine serum (FBS) supplemented with GlutaMax (ThermoFisher) at 37° C. with 5% CO₂.

### Construction of miR-96 s1334 Cell Line Using PiggyBac

Mouse miR-96 s1334 (˜0.6 kb) harboring the +14C>A mutation was amplified by PCR from miR-96^{s1334/s1334} mouse genomic DNA. The PCR products were cloned into the PiggyBac donor backbone (PB-CAG-mNeonGreen-P2A-BSD-polyA) using Gibson Assembly. The constructed donor plasmid was co-transfected with PiggyBac transposon vector (PB210PA, System Biosciences) into HEI-OC1 cells. Cells were cultured and selected in the medium containing 10 μg/mL Blasticidin for 2 weeks. For human miR-96+14C>A fragment, mutation was introduced by PCR and cloned into the same PiggyBac donor backbone using Gibson Assembly. Cells were transfected by PiggyBac plasmids and selected by Blasticidin for 2 weeks. Successful insertion was confirmed by PCR and sequencing analysis. Clones from the positive miR-96 s1334 selection were expanded for subsequent studies.

### Genome Editing In Vitro

For transfection of fibroblasts, nucleofection was performed using LONZA 4D-Nucleofector. Cells were digested by Trypsin-EDTA (0.05%) (Thermo Fisher) for 6-8 minutes and dispersed into single cells. After washing with PBS, about 100,000 cells were resuspended in 20 μl P3 reagent of the P3 Primary Cell 4D-Nucleofector® X Kit S (Lonza V4XP-3032). 1 μg total plasmid was used for a single nucleofection event and nucleofected by program EH-100. Cells were not sorted. 5 days after the nucleofection, cells were collected and lysed by QuickExtract™ DNA Extraction Solution (Lucigen) and incubated in 65° C. for 10 min then 98° C. 3 min to extract genome DNA. Genomic DNA was isolated and analyzed by Sanger sequencing cell lines for PCR genotype. Genomic PCR was carried out using NEBNext® Ultra™ II Q5® Master Mix (NEB, M0544S) to amply miR-96 loci, PCR products were visualized on a 2% agarose gel using GelRed (Biotium) and purified on a column (QIAquick Gel Extraction Kit, Qiagen). 400˜800 ng of purified PCR product were used for next generation sequencing (NGS), samples were sequenced and analyzed to detect CRISPR variants from NGS reads using a custom algorithm developed by the Massachusetts General Hospital Center for Computational and Integrative Biology DNA Core (MGH DNA core). Individual SEQ files were analyzed by CRISPResso2 according to the program manual. The sgRNA protospacer sequences can be found in Table 1.

### Hair Cells Isolation and NGS Analysis

Cochleae were harvested with the sensory epithelia dissociated using needles under the microscope (Axiovert 200M, Carl Zeiss). Inner ear tissue was immersed in 1 μM FM 1-43FX (ThermoFisher, F35355) dissolved in DPBS (ThermoFisher) for 15 s at room temperature in the dark, then washed by DPBS. The sensory epithelia were treated with 100 μl 0.05% trypsin-EDTA (ThermoFisher, 25300054) for 5-10 min. During incubation, the tissue was dispersed into small cell clusters or single cells using a 200 μl Eppendorf pipette tip. Cells were then transferred into a well of 24-well plate and placed under a fluorescent microscope (ZEISS) equipped with a camera. Cells with FM 1-43FX dye were collected by a 10 μl Eppendorf pipette tip. About 300 cells were collected from single cochlea. Hair cells from three cochleae were transferred into 200 μl PCR tube, and centrifuged for 5 min, 200 rcf. Supernatant were carefully discarded and the isolated hair cells were lysed by 5 μl QuickExtract™ DNA Extraction Solution (Lucigen) and incubated in 65° C. for 6 min then 98° C. 3 min. All 5 μl of the cell lysis were used for each Genomic PCR amplification using NEBNext® Ultra™ II Q5® Master Mix (NEB, M0544S). PCR program is 1 cycle: 98° C. 5 min; 42 cycles: 98° C. 15 s, 60° C. 20 s, 72° C. 10 s; 1 cycle: 72° C. 4 min; 4° C. PCR products visualization, purification and NGS analysis are the same with that described above.

### RNA Isolation and qRT-PCR

Total RNA was extracted from inner eat tissue using the ReliaPrep RNA Tissue Miniprep System (Promega, z6111). Then first-strand cDNA was produced using ProtoScript® II First Strand cDNA Synthesis Kit (NEB, E6560s) with random primers, following the manufacturer's instructions. STEM-LOOP qRT-PCR were used to measure miR-96 level, first-strand cDNA was produced using miR-96 specific primers (rtmri96: CTCAACTGGTGTCGTGGAGTCGGCAATTCAGTTGAGCAAAAATGTG). Real time quantitative PCR was performed using Power SYBR Green PCR Master Mix (Applied Biosystems, 4368708) on the ABI QuantStudio 3 Flex Real-Time PCR System (Applied Biosystems). qmiR96-F: TCGGCAGGTTTGGAACTAGCAC; qmiR96-R: CTCAACTGGTGTCGTGGA.

### Western Blotting

After dissection of cochlea, inner ear tissue samples were lysed using by RIPA Lysis and Extraction buffer (ThermoFisher, 89900) containing protease inhibitor (ThermoFisher) for 30 min on ice. Protein was quantified and 80 μg of each lysate were loaded per lane of a NuPAGE™ 4-12% Bis-Tris Protein Gel (Thermo Fisher Scientific, NP0335PK2). Samples were separated on 200V for 35 min in the mini gel tank (ThermoFisher). Protein were then transferred to Nitrocellulose Blotting Membranes (PALL, P66485) at 200 mA for 2 hours. The membranes were blocked in 5% evaporated milk in Tris-based saline with Tween 20 (0.05% TBST) for 1 hour and followed by incubating with primary antibodies overnight at 4° C. (Anti-HA: Cell Signaling Technology, 3724S; anti-GAPDH: Thermo, MA5-15738). NC membranes were then washed three times with TBST and incubated with HRP conjugated secondary antibodies (Thermo Fisher Scientific) for 2 hours at RT. Protein bands were visualized using SuperSignal West Pico PLUS Chemiluminescent Substrate (ThermoFisher, 34577) and blot image were captured by ChemiDoc Imaging system (BioRad).

### AAV Vector Integration Assay

HEK 293T-miR96-s1334 cells were treated with AAV2-CMV-KKH-saCas9-sgRNA4 of different dosages from 1 to 107 genomic copies per cell. Control cells were transduced with AAV2-CMV-KKH-saCas9-sgCtrl, 10⁵ genomic copies per cell. Cells were collected 7 days later and genomic DNA was isolated. Primers P1-F/ITR-R were used for detecting of AAV vector integration. For in vivo integration detection, Primers P1-F/P2-R and primers P1-F/ITR-R were used to amply genomic fragment from isolated hair cells, then PCR products were merged together for NGS analysis. Primers premir96-F/ITR-R were used in the qRT-PCR study to detect mir96-ITR transcription.

### Off-Target Analysis

For each sgRNA, the top 10 potential off-target sites according to the cutting frequency determination (CFD) score. PCR primers were designed on the two flanks of each potential sgRNA target sequence for amplifying 250-280 bp DNA fragments. Then amplified fragments were purified for NGS to identify whether there was any off-target mutation after editing.

### Example 1: Screening of Genome Editing Systems Targeting miR-96 +14 C>A Domo Loci

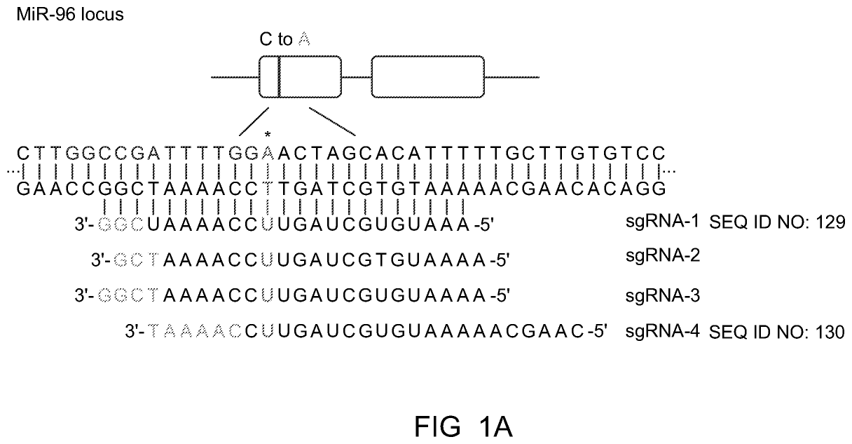

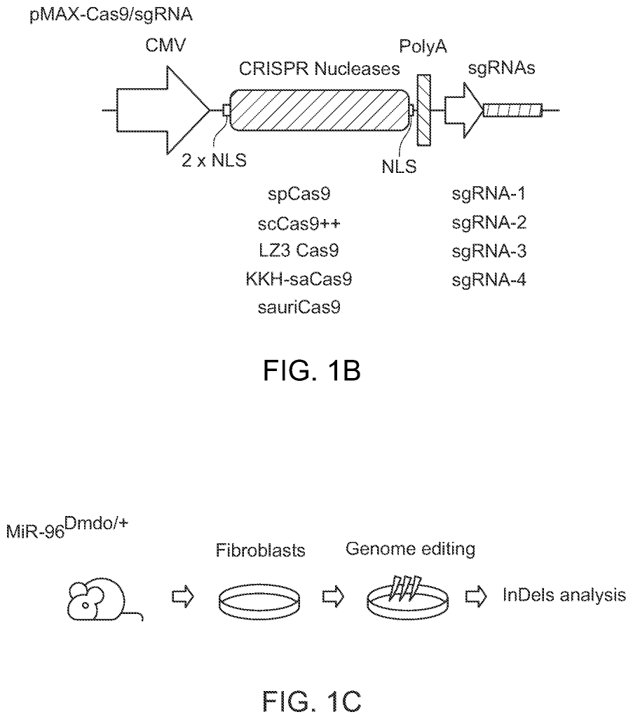

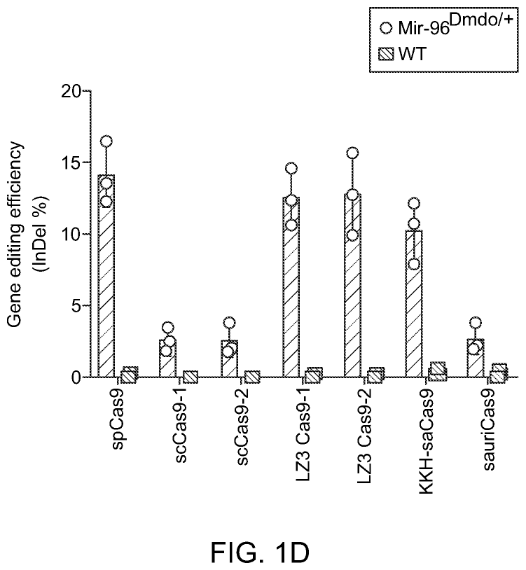

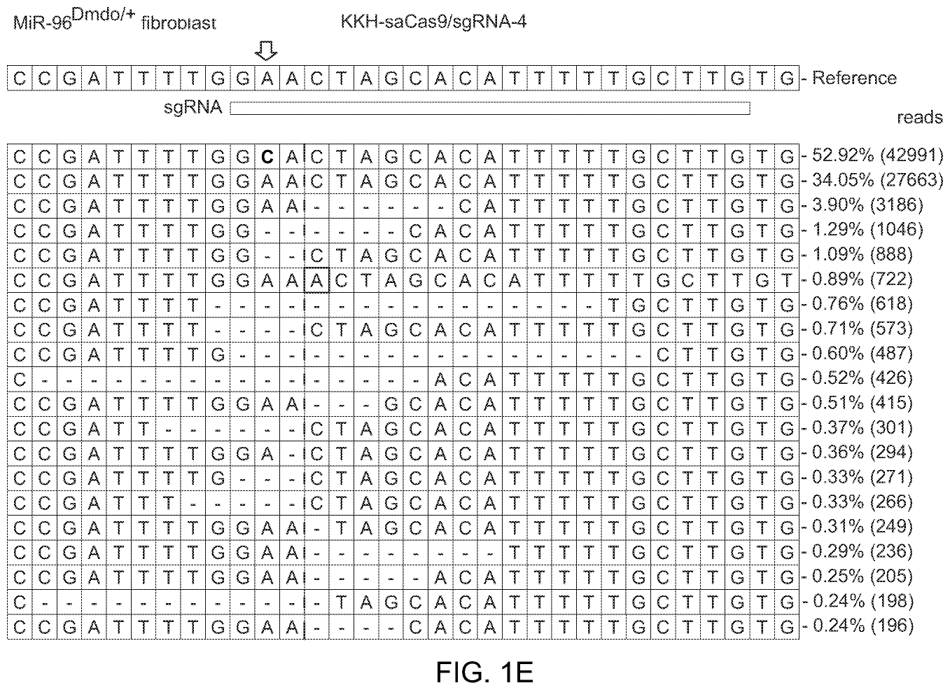

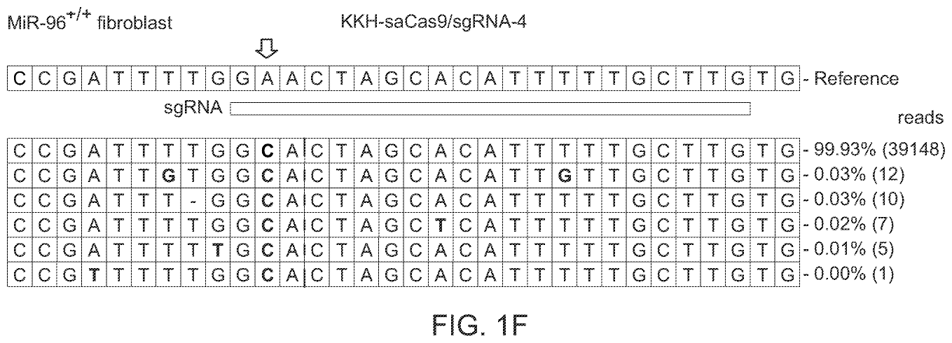

In order to selectively disrupt the miR-96 Dmdo allele (depicted in FIG. 1A), four sgRNAs were designed to target the miR-96 Dmdo allele in combination with different Cas9 nucleases (FIG. 1B). The Dmdo allele is also known as the s1334allele and is these terms are used interchangeably herein to refer to mouse strains bearing mutations of the mir96 locus. Combinations of the four sgRNAs and various Cas9 nucleases were screened for editing efficiency in miR-96^{Dmdo/+} mice primary fibroblasts (experimental design depicted in FIG. 1C). As shown in FIG. 1D, sgCas9/sgRNA-1 showed the highest editing efficiency: 14.3±2.3%. LZ3 Cas9/sgRNA-1 and KKH-saCas9/sgRNA-4 also have comparable editing efficiencies of 12.7±2.0% and 10.1±2.1%, respectively. As shown in FIG. 1E, in mice primary fibroblasts, nearly all indels that occurred in miR-96^{Dmdo/+} cells were present just in the mutant allele and indels were almost undetectable in miR-96^{+/+} cells (less than 0.1%, FIG. 1F).

### Example 2: Efficient and Specific Genome Editing at miR-96 14 C-to-A Locus in Human Cells

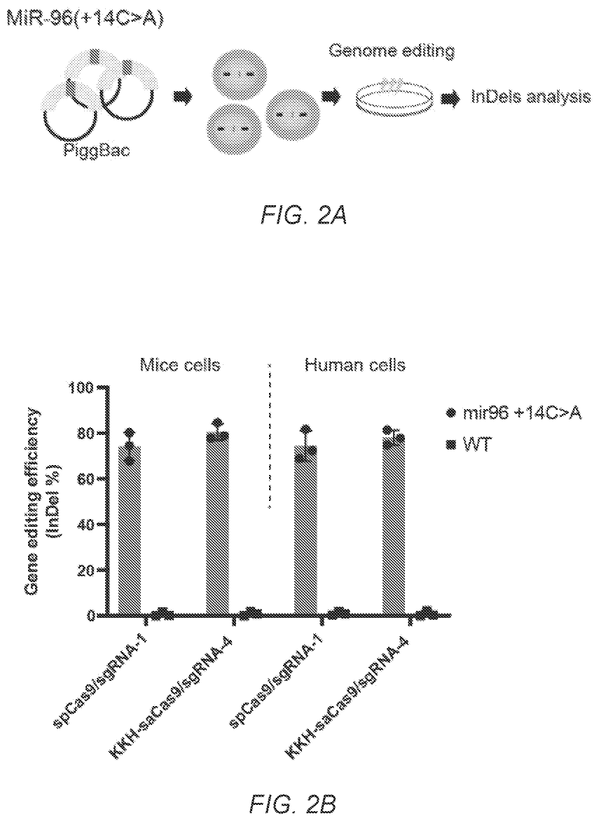

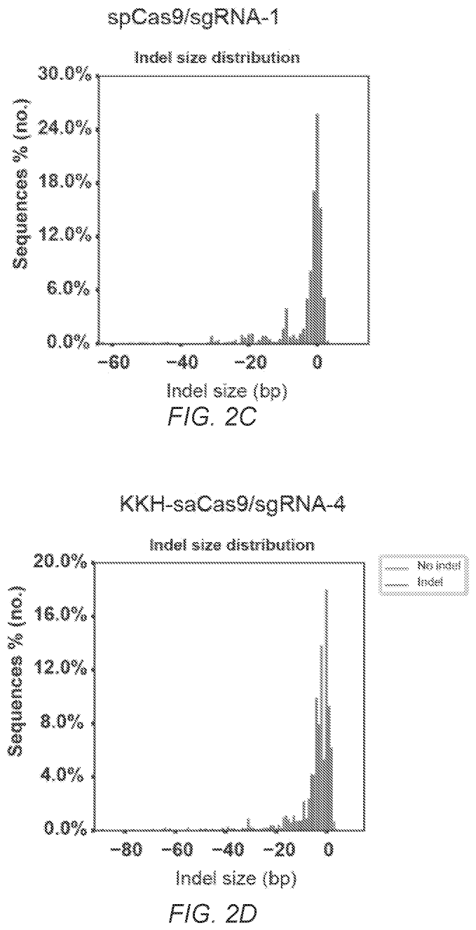

As the nucleotide sequence of the miR-96 locus is fully conserved between mouse and human, the same sgRNAs targeting miR-96 Dmdo allele can also be applied for editing the human miR-96 locus. As depicted in FIG. 2A, a human cell line was engineered to contain the human miR-96 C-to-A mutation, and the cells were transfected with spCas9/sgRNA and KKH-saCas9/sgRNA targeting the mutant locus. As shown in FIG. 2B, indel analysis revealed robust genome editing in miR-96 14 C-to-A loci, but not in wild-type loci. NGS data revealed many different types of indels for both spCas9/sgRNA and KKHsaCas9/sgRNA. As shown in FIGS. 2B-2C, the most common indel event was a single nucleotide deletion for spCas9/sgRNA-1 and a six nucleotide deletion for KKH-saCas9/sgRNA-4.

### Example 3: Optimization of AAV Design to Increase Editing Efficiency

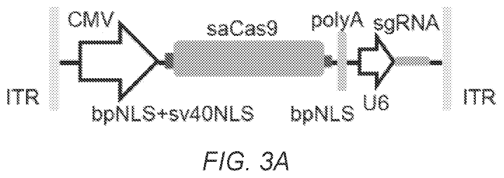

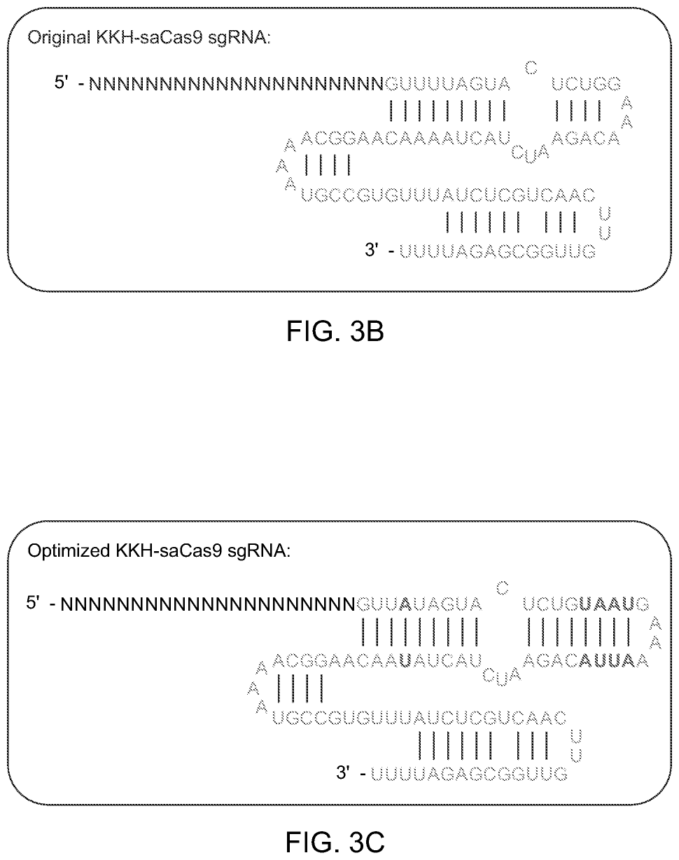

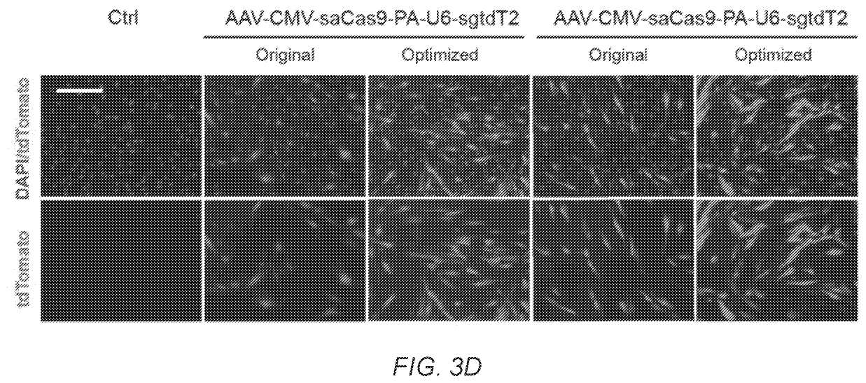

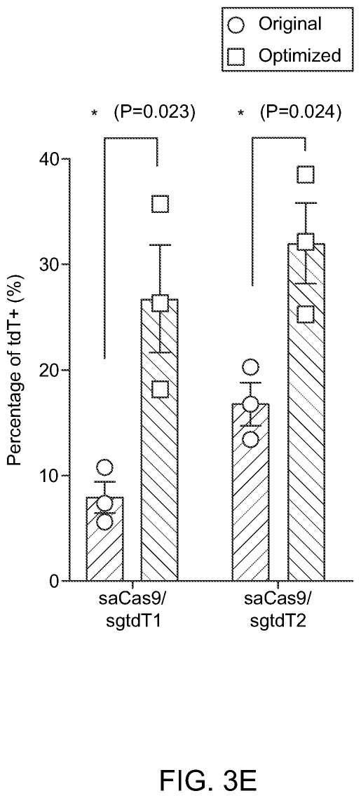

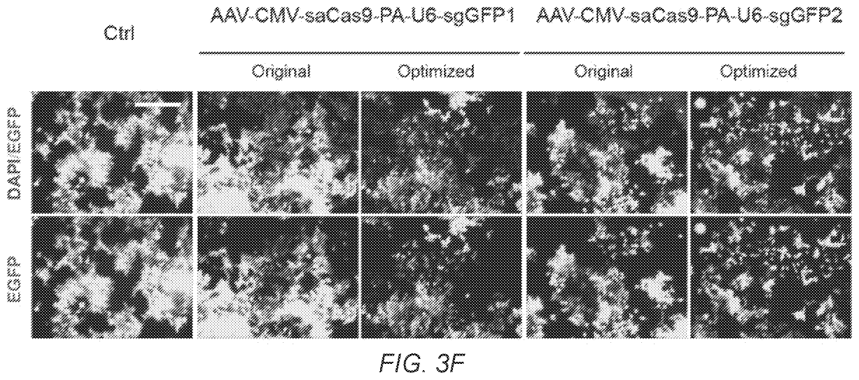

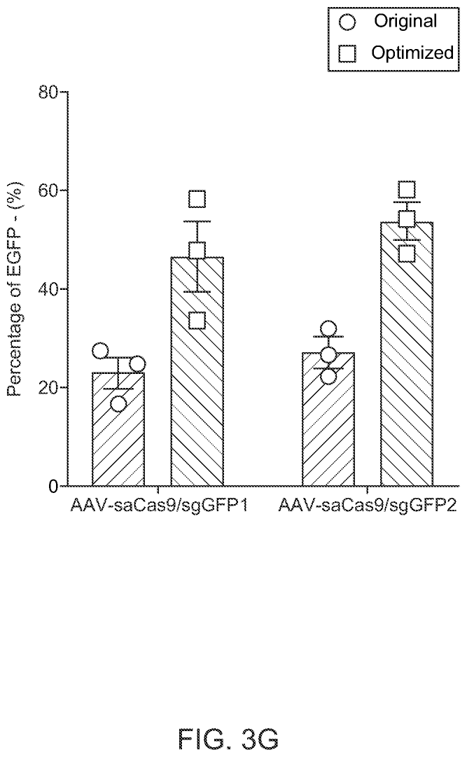

Before packaging the combinations of Cas9/sgRNA into AAVs, AAV constructs were optimized in order to improve the editing efficiency after delivery to cells. For the KKH-saCas9/sgRNA system, one bipartite nuclear localization signal (bpNLS) and one SV40 NLS were added to the N-terminus of the saCas9 and one bpNLS was added at the C-terminus (shown in FIG. 3A). Further, as shown in FIGS. 3B-3C, the sgRNA was optimized to enhance editing efficiency (nucleotide substitutions highlighted in bold in FIG. 3C). Editing efficiency was evaluated in primary fibroblasts derived from Ai9 mice. Ai9 mice harbor a Rosa-CAG-LSL-tdTomato-WPRE conditional allele. A loxP-flanked STOP cassette is designed to prevent transcription of the red fluorescent protein variant tdTomato. When bred to mice that express Cre recombinase, the resulting offspring will have the STOP cassette deleted in the cre-expressing tissue(s)-resulting in robust tdTomato fluorescence. In this assay, using primary fibroblasts derived from Ai9 mice, tdTomato fluorescence indicates edited cells. As shown in FIGS. 3D-3E, the optimized AAV constructs delivered higher editing efficiency than the original constructs. Additionally, editing efficiency was measured in HEK-GFP cells targeting GFP. As shown in FIGS. 3F-3G, GFP negative cell ratio also indicated higher editing efficiency using optimized AAV constructs.

### Example 4: In Vivo Genome Editing at miR-96 Domo Allele in Adult miR-96^{Dmdo/+} Cochlea

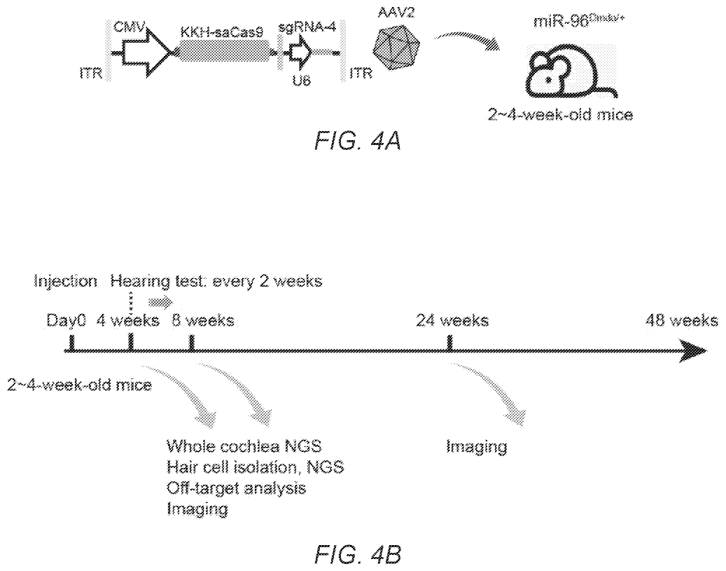

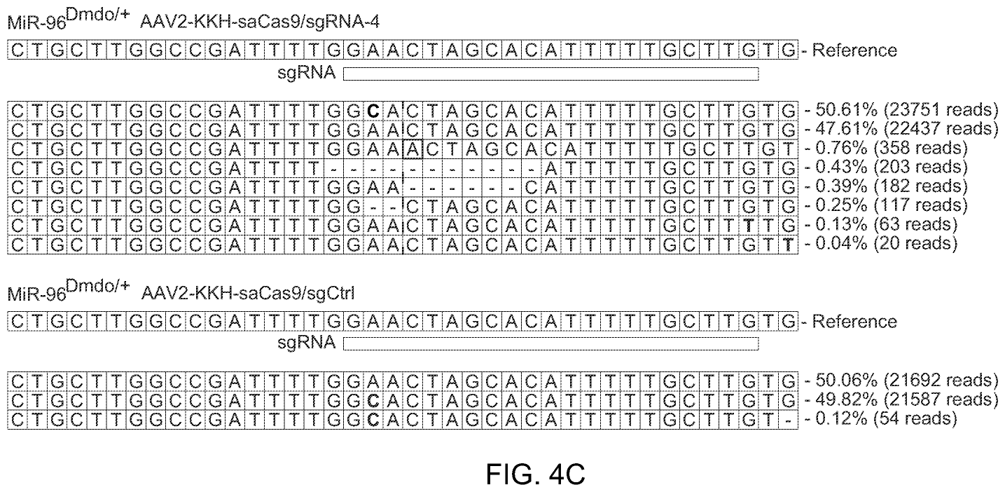

As shown in the schematic of FIG. 4A, KKH-saCas9/sgRNA-4 were packaged into an AAV2 vector and injected into adult miR-96^{Dmdo/+} cochlea at 2-4 weeks of age using round window membrane and canal fenestration (RWM+CF) injection. The experimental design is represented in the schematic of FIG. 4B. In vivo genome editing efficiency was evaluated two months after the injection. As shown in the representative sequence reads of FIG. 4C, NGS from whole cochlear tissue showed evidence of genome editing at miR-96 mutant loci in injected mice, but not in sgCtrl group. The AAV2-KHH-saCas9-sgRNA-4 composition was specific for the miR-96 Dmdo allele, as no indels were observed in wild-type control mice.

### Example 5: In Vivo Editing at miR-96 Domo Locus Preserves Outer Hair Cells (OHCs) Survival and Rescues Hearing

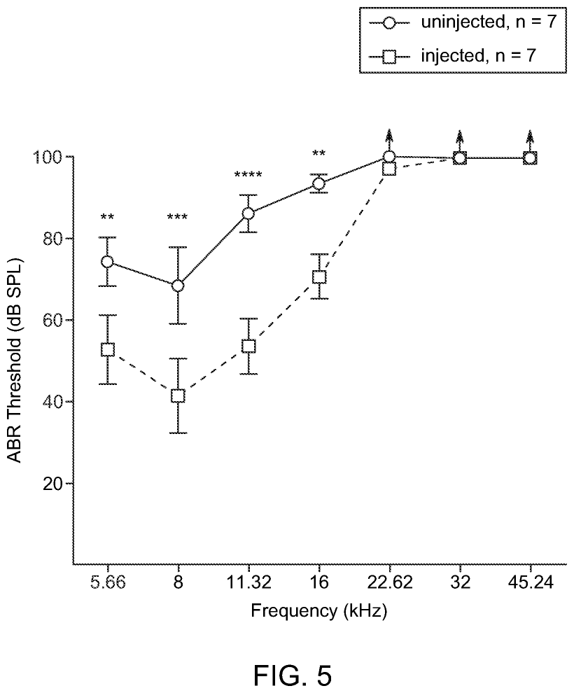

miR-96 expression is first detected in the otic vesicle at E9.5 during mouse embryonic development and exhibits strong expression in the cochlear hair cells during hair cell development. The expression continues in adult mice, playing an important role in developing and maintaining normal hearing. The AAV2-KKH-saCas9-sgRNA-4 composition was unilaterally injected into cochlea of adult miR-96^{Dmdo/+} mice at 2-4 weeks of age. The auditory brainstem response (ABR) hearing test was performed 3.5 months after injection. As shown in FIG. 5, results of the ABR test indicate an improvement in hearing at low frequency ranges relative to un-treated contralateral ear of the same animal.

### Example 6: Precise Detection of the Editing Efficiency in Hair Cells after Injection

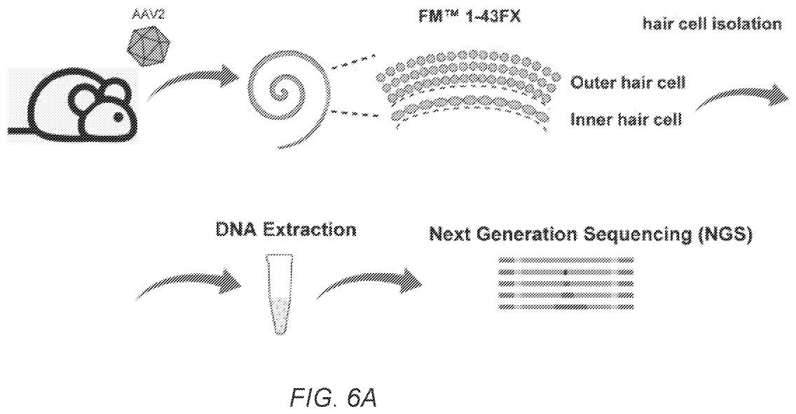

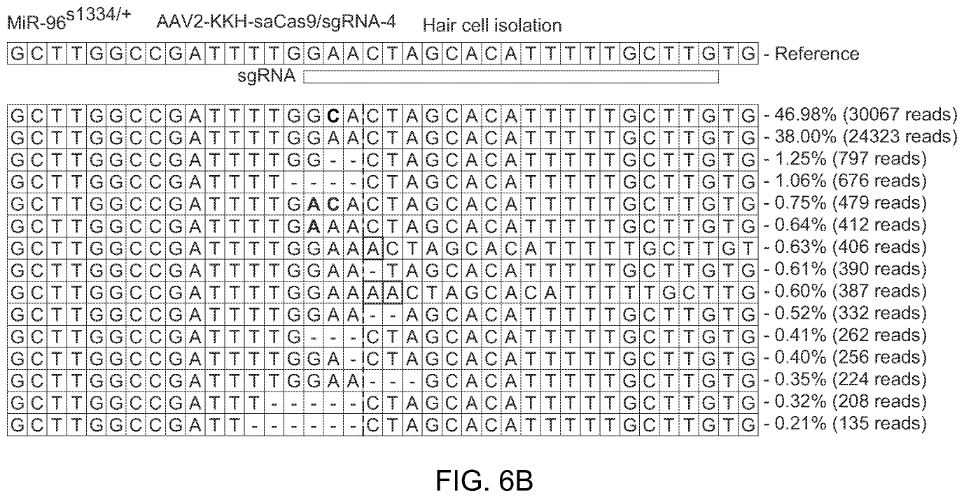

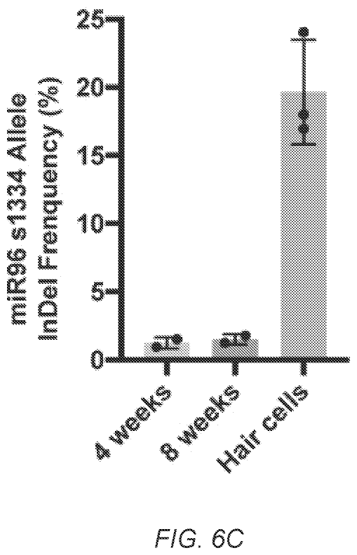

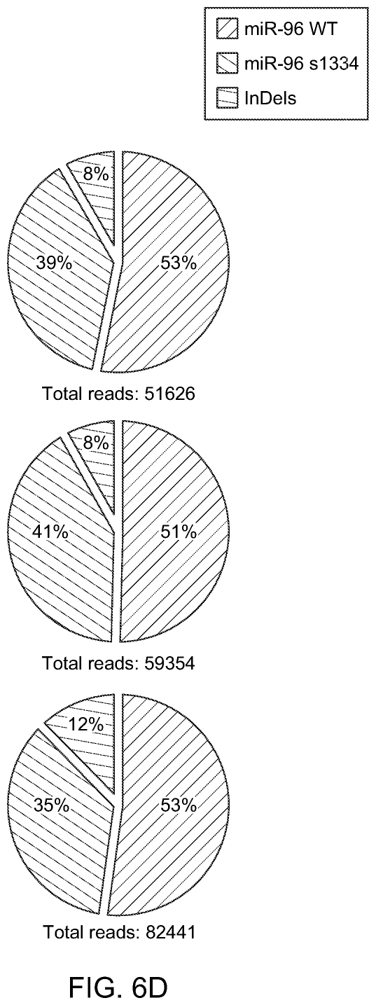

The indel frequency as measured in whole organ of Corti samples may not accurately reflect the editing efficiency in the subpopulation of hair cells, as the indel rates of the cochlea tissue may be masked by genomic DNA extracted from other types of cells such as supporting cells, which vastly outnumber hair cells in the whole organ. To obtain a precise measurement of the editing efficiency in hair cells specifically, hair cells were isolated using the FM1-43 uptake assay. FM1-43, which is a fluorescent dye, and its fixable analog FM1-43FX, can specifically label the hair cells by passing through open mechanotransduction channels. As shown in the experimental design of FIG. 6A, after FM1-43FX treatment, the labeled cells were isolated and lysed, then the indel frequency was analyzed by NGS in isolated hair cells. Indel-containing miR-96 sequencing reads from miR-96^{s1334/+} mice injected with the AAV2-KHH-saCas9-sgRNA-4 composition allowed for quantification of the specificity of editing of the miR-96 s1334 allele in vivo. As shown in FIG. 6B, in contrast to the uninjected ears, robust indel formation was observed in isolated cells from the injected animals eight weeks after treatment. As shown in the plot of FIG. 6C, the indel frequency was about 16.3-25.7% in miR-96 s1334 allele. Also, a wider range of indel types were observed in the isolated hair cells relative to the indels observed in whole cochlea samples, indicating efficient targeted genome editing of the miR-96 locus specifically in hair cells. In three independent experiments, the ratio of miR-96 wild-type allele reads vs s1334 allele reads was analyzed by NGS after editing. As shown in FIG. 6D, in the injected ear, the ratio of miR-96 s1334 unedited reads combined with indel-containing reads is nearly the same as that of miR-96 wild-type reads (48±1% vs. 52±1%), indicating no significant chromatin lesion or insertion caused by genome editing.

### Example 7: A Dual AAV2 System Enables Targeting Multiple Human miR-96 Mutations

The miR-96 nucleotide sequence is conserved throughout vertebrate evolution and the Cas9/sgRNAs compositions disclosed herein successfully target miR-96 mutations in mouse cells and human cells. To further evaluate editing of the miR-96 locus, two additional disease-associated dominant mutations in miR-96 seed region were identified, and sgRNAs were designed to target these mutations in addition to the 14 C-to-A mutation. As shown in FIG. 7A, miR-96+14 C>A, +13 G>A, and +15 A>T were targeted. As shown in FIG. 7B, a dual-AAV system was designed to target all three miR-96 mutations simultaneously with a single administration. The dual-AAV system includes AAV-U1A-spCas9-polyA with 3 NLSs to expression the nuclease, and AAV-sgmiR96-master with three sgRNA cassettes, one cassette targeting each of the three miR-96 mutations. Further, as shown in FIG. 7C, the spCas9 was optimized to enhance editing efficiency. To validate the dual AAV system, multiple human cell lines were engineered to contain each of the three different miR-96 mutations. Cells were then transfected with AAV-U1A-spCas9-polyA and AAV-sgmiR96-master, DNA was extracted, and NGS was performed to evaluate editing efficiency. As shown in FIG. 7D, NGS results indicate indel formation in all three miR-96 mutation lines with similar efficiency. As shown in FIG. 7E, indels were observed in less than 1% of reads generated from the miR-96 wild-type locus, suggesting specific disruption of the mutant allele. FIGS. 7F-7G show plots of indel profiles from spCas9/sgmiR96-master edited HEK-miR96 (+15 A>T) cells. Negative numbers represent deletions, positive numbers represent insertions. Indel frequency for each of three mutations and wild-type was quantified and plotted in FIG. 7H. To validate the specificity of the dual AAV system, editing at the top ten off-target hits according to the cutting frequency determination (CFD) score was evaluated in human cells. As shown in FIG. 7I, after Cas9/sgRNA transfection with AAV-U1A-spCas9-polyA and AAV-sgmiR96-master, no indels were detected in the top ten predicted potential off-target sites. These results indicate that the combination of AAV-U1A-spCas9-polyA and AAV-sgmiR96-master successfully target three disease-associated miR-96 mutations in human cells and may be useful for treating miR-96 related non-syndromic progressive hearing loss. The design of dual AAV sgmiR96 master system broadens the targeting scope to multiple mutations with a single administration and simplifies the efficacy and safety evaluation of the therapeutic AAV delivery system.

### Example 8: In Vivo Genome Editing Preserves Hair Cell Survival

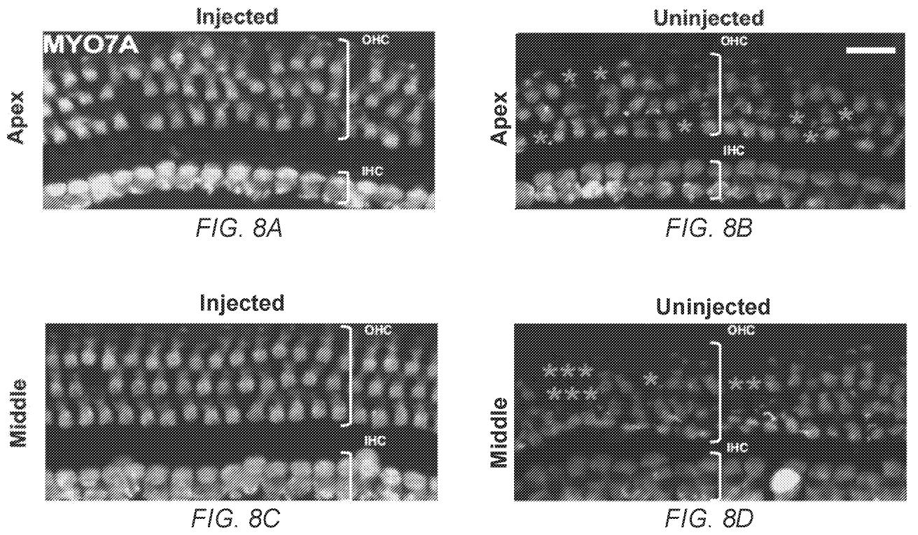

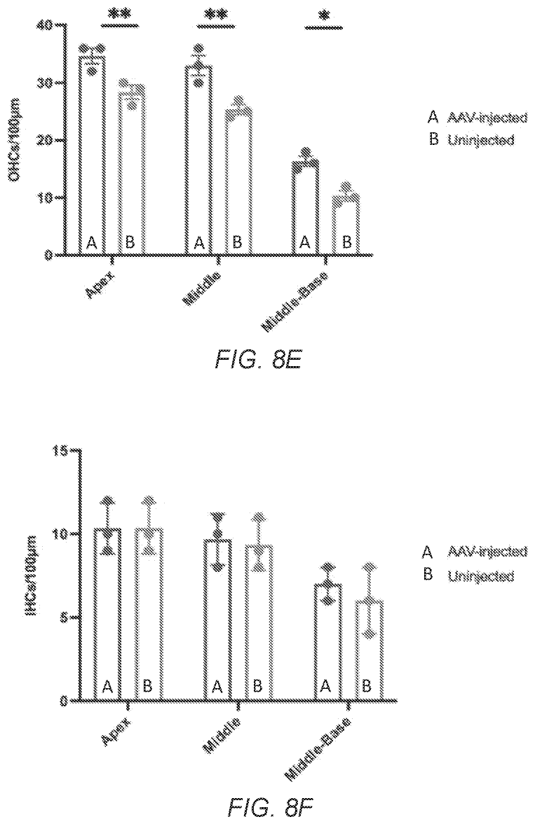

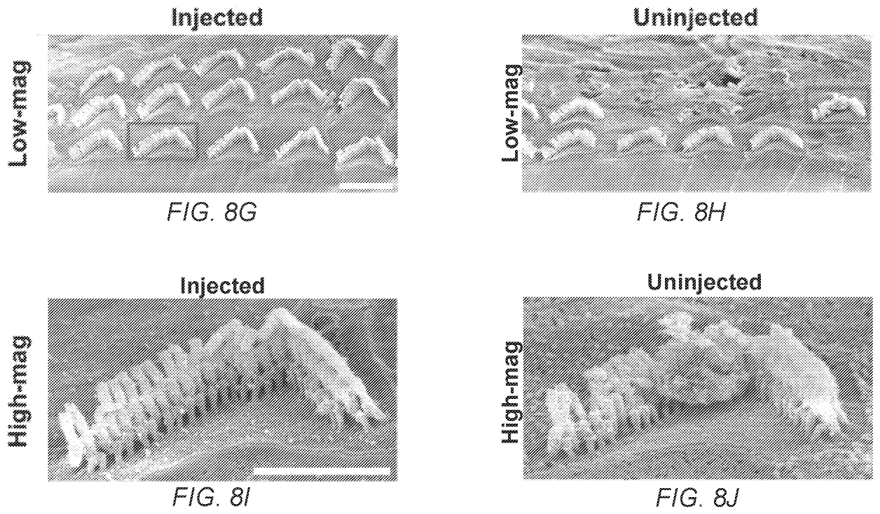

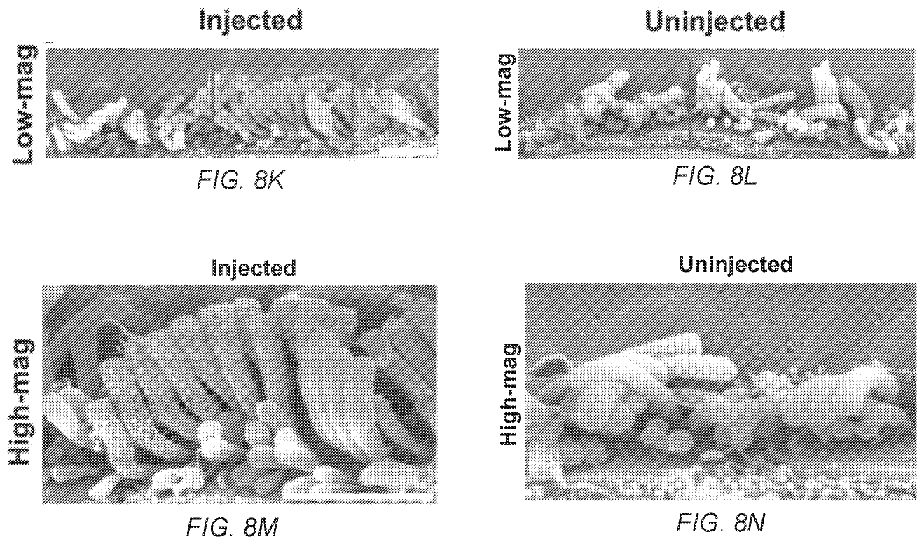

Homozygous Dmdo mice, miR-96^{Dmdo/Dmdo}, and homozygous null mice, miR-96^{-/-}, are completely deaf, with abnormal hair cell stereocilia bundles and reduced number at early age, indicating developmental dysfunction. In order to evaluate the effect of disrupting the mutant mir96 locus by administering the genome editing system disclosed herein, the AAV2-KKH-saCas9-sgRNA-4 composition was injected into adult miR-96s^{1334/+} mouse cochlea and hair cell morphology was evaluated by scanning electron microscopy. FIG. 8A is a representative confocal microscopy whole mount image of AAV2-KKH-saCas9-sgRNA-4 injected miR-96s^{1334/+} mouse cochlea, featuring the apex. Cochlea was stained with MYO7A. FIG. 8B is a representative confocal microscopy whole mount image uninjected negative control miR-96s^{1334/+} mouse cochlea, featuring the apex. Cochlea was stained with MYO7A.

Degeneration of hair cells is indicated by asterisks. FIG. 8C is a representative confocal microscopy whole mount image of AAV2-KKH-saCas9-sgRNA-4 injected miR-96s^{1334/+} mouse cochlea, featuring the middle of the cochlea. Cochlea was stained with MYO7A. FIG. 8D is a representative confocal microscopy whole mount image uninjected negative control miR-96s^{1334/+} mouse cochlea, featuring the middle of the cochlea. Cochlea was stained with MYO7A. Degeneration of hair cells is indicated by asterisks. FIG. 8E is a plot of the number of outer hair cells (OHCs) per 100 μm section for three untreated and three AAV2-KKH-saCas9-sgRNA-4 treated miR-96s^{1334/+} cochleae from three mice at 10 weeks after treatment. Values and error bars reflect the mean±standard deviation. FIG. 8F is a plot of the number of inner hair cells (IHCs) per 100 μm section for three untreated and three AAV2-KKH-saCas9-sgRNA-4 treated miR-96s^{1334/+} cochleae from three mice at 10 weeks after treatment. Values and error bars reflect the mean±standard deviation. FIG. 8G is a representative scanning electron microscopy (SEM) image of AAV2-KKH-saCas9-sgRNA-4 injected miR-96^{s1334/+} outer hair cell bundles at apex region. FIG. 8H is a representative scanning electron microscopy (SEM) image of uninjected negative control miR-96^{s1334/+} outer bair cell bundles at apex region. Degenerated stercocilia are indicated by asterisks.

These results indicate the disrupting the mutant miR86 allele by in vivo administration of the genome editing system disclosed berein preserves hair cell survival in adult mammalian cochlea.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising one or more nucleic acids comprising a sequence encoding an RNA-guided nuclease and a sequence encoding one or more single guide RNAs (sgRNAs), wherein the target sequence of the one or more sgRNAs comprises a mutation of the miR-96 locus selected from the group consisting of +14 C>A, +13 G>A, and +15 A>T relative to SEQ ID NO: 172.

2. The composition of claim 1, wherein the target sequence of the one or more sgRNAs comprises any one of SEQ ID NOs 1-167.

3. The composition of claim 1, wherein the RNA-guided nuclease is a Cas9 nuclease.

4. The composition of claim 3, wherein the Cas9 nuclease is selected from the group consisting of spCas9 or variant thereof, saCas9 or variant thereof, scCas9++, LZ3 Cas9, KKH-saCas9 and sauriCas9.

5. The composition of claim 1, wherein the one or more sgRNAs each further comprises a sequence selected from any one of SEQ ID NOs. 168-171.

6. The composition of claim 1, wherein the RNA-guided nuclease comprises one or more nuclear localization signals.

7. The composition of claim 6, wherein the one or more nuclear localization signals comprise a C-terminal nuclear localization signal and/or an N-terminal nuclear localization signal

8. The composition of claim 1, wherein the sequence encoding the RNA-guided nuclease comprises a polyadenylation signal.

9. The composition of claim 1, wherein the one or more nucleic acids is a viral delivery vector.

10. The composition of claim 9, wherein the viral delivery vector is an adenovirus vector, an adeno-associated virus (AAV) vector, or a lentivirus vector.

11. The composition of claim 1, wherein a first nucleic acid comprises the sequence encoding the RNA-guided nuclease and a second nucleic acid comprises the sequence encoding the one or more sgRNAs.

12. The composition of claim 11, wherein the second nucleic acid comprises:
(i) a first sgRNA that targets the +14 C>A mutation of the miR-96 locus;
(ii) a second sgRNA that targets the +13 G>A mutation of the miR-96 locus; and
(iii) a third sgRNA that targets the +15 A>T mutation of the miR-96 locus.

13. The composition of claim 12, wherein the first sgRNA comprises SEQ ID NOs: 129 and 171, the second sgRNA comprises SEQ ID NOs: 127 and 171, and the third sgRNA comprises SEQ ID NOs: 128 and 171.

14-17. (canceled)

18. A composition comprising a ribonucleoprotein (RNP) complex comprising an RNA-guided nuclease and an sgRNA, wherein the target sequence of the sgRNA is any one of SEQ ID NOs 1-167.

19. The composition of claim 18, wherein the Cas9 nuclease is selected from the group consisting of spCas9, scCas9++, LZ3 Cas9, KKH-saCas9 and sauriCas9.

20. The composition of claim 18, wherein the sgRNA comprises:
(i) SEQ ID NOs: 129 and 171;
(ii) SEQ ID NOs: 127 and 171; or
(iii) SEQ ID NOs: 128 and 171.

21. A method of disrupting a mutant allele of the miR-96 locus in a cell, the mutant allele being selected from the group consisting of +14 C>A, +13 G>A, and +15 A>T relative to SEQ ID NO: 172, further comprising contacting the cell with the composition of claim 1.

22. The method of claim 21, wherein disrupting the mutant allele is effected using a sgRNA having a target sequence of any one of SEQ ID NOs 1-167.

23. The method of claim 21, wherein the cell is in or from a subject who has non-syndromic progressive hearing loss.

24. The method of claim 21, wherein the cell is a cell of the inner ear of the subject.

25. The method of claim 24, wherein the cell is an outer hair cell.

26. A method of treating progressive non-syndromic hearing loss in a patient in need thereof, the method comprising administering to the patient the composition of claim 1.

27. The method of claim 26, wherein the patient harbors a mutation of the miR-96 locus selected from the group consisting of +14 C>A, +13 G>A, and +15 A>T relative to SEQ ID NO: 172.
